(19) United States
(12) Patent Application Publication
(10) Pub. No.: US 2024/0277393 A1
(43) Pub. Date: Aug. 22, 2024

(54) ELECTROPORATION TREATMENT

(71) Applicant: Entire Medical Inc., Fort Lauderdale, FL (US)

(72) Inventors: Mordechay BEYAR, Tel-Aviv (IL); Oren GLOBERMAN, Kfar-Shemaryahu (IL); Danielle VALES, Ramat HaSharon (IL)

(73) Assignee: Entire Medical Inc., Fort Lauderdale, FL (US)

(21) Appl. No.: 18/570,657

(22) PCT Filed: Jun. 16, 2022
(86) PCT No.: PCT/IL2022/050649

§ 371 Date: Dec. 15, 2023

Related U.S. Application Data
(60) Provisional application No. 63/211,022, filed on Jun. 16, 2021.

(51) Int. Cl. A61B 18/00

(52) U.S. Cl. CPC A61B 18/00; A61B 2018/00083; A61B 2018/00327; A61B 2018/00577; A61B 2018/00613; A61B 2018/00714; A61B 2018/00732; A61B 2018/00761; A61B 2018/00767; A61B 2018/0091; A61B 2218/007

(57) ABSTRACT

The invention relates to a method of reducing volume of a tissue without damaging epithelial tissue, the method comprising inserting an IRE device within a zone of a mouth or a nose, enclosing an area of interest of said tissue within a space between at least two electrodes; and applying irreversible electroporation ablation treatment.

### RELATED APPLICATION/S

This application claims the benefit of priority of U.S. Provisional Patent Application No. 63/211,022 filed on 16 Jun. 2021, the contents of which are incorporated herein by reference in their entirety.

### FIELD AND BACKGROUND OF THE INVENTION

The present invention, in some embodiments thereof, relates to electroporation treatments and, more particularly, but not exclusively, to non-invasive electroporation treatments.

Irreversible electroporation (IRE) is a tissue ablation technique using short but strong electrical fields to create permanent and hence lethal nanopores in the cell membrane, to disrupt cellular homeostasis. The resulting cell death results from induced apoptosis or necrosis induced by either membrane disruption or secondary breakdown of the membrane due to transmembrane transfer of electrolytes and adenosine triphosphate. One of the main uses of IRE lies in tumor ablation in regions where precision and conservation of the extracellular matrix, blood flow, and nerves are of importance.

Additional background art includes U.S. Patent Application No. US2016022989AA disclosing a non-user controllable electro-therapy device having a housing for a microprocessor, power source, status indicator, activation switch, and one or more channels for electrode contact. Only the activation switch is user-accessible. The microprocessor generates a non-user controllable frequency-dependent mixed electrical signal through the electrodes, wherein the mixed electrical signal is a combination of at least two different frequencies, a first frequency having a first minimum and maximum microamp range and a second frequency having a different second minimum and maximum microamp range. The higher of the two frequencies are superimposed on the lower frequency, creating a current intensity window as an envelope along with a profile of the lower frequency. The mixed electrical signal is automatically applied for a pre-determined period of time, and amplitude and/or duration, and/or frequencies are varied according to a pre-set schedule programmed into a controller coupled to one or more electrodes.

U.S. Patent Application No. US2013041310AA disclosing electroporation (EP) devices that are able to generate electroporation causing electrical field at the mucosal layer, and preferably in a tolerable manner. Further, it includes the generation of protective immune response, cellular and/or humoral, using the oral EP device along with a genetic construct that encodes an immunogenic sequence.

U.S. Patent Application No. US2020205892AA disclosing systems, devices, and methods for electroporation ablation therapy, with the system including a pulse waveform signal generator for medical ablation therapy, and an endocardial ablation device including an inflatable member and at least one electrode for focal ablation pulse delivery to tissue. The signal generator may deliver voltage pulses to the ablation device in the form of a pulse waveform. The system may include a cardiac stimulator for the generation of pacing signals and for sequenced delivery of pulse waveforms in synchrony with the pacing signal.

U.S. Patent Application No. US2013261683AA discloses a method, device, and system which employs particles, such as nanoparticles, and an electric or electromagnetic field, to cause cell death in target cells by non-thermal means. The method of causing targeted cell death comprises the steps of introducing a particle to the interior of a target cell and exposing the target cell to a transient electromagnetic field for a sufficient time interval in order to cause cell death. The invention overcomes problems associated with similar methods as a result of the fact that a smaller electric field is applied because the particle enhances the effect of the electric field in its immediate vicinity, so reducing the field strength needed to achieve cell lysis and thereby reducing the risk of damage to healthy cells that may be in its vicinity. Apparatus for performing the method; as well as techniques of delivering particles and for producing particles are also described.

U.S. Patent Application No. US2013261683AA disclosing a method, apparatus, and system that employs particles, e.g., nanoparticles, and an electric or electromagnetic field, to cause electroporation in target cells at reduced fields. Electroporation may be irreversible, leading to targeted cell death, or reversible, allowing species to be introduced into the target cell. The method introduces a particle to a position adjacent to the cell membrane of a target cell and exposes the target cell to a transient electromagnetic field for a time interval to cause targeted electroporation. A smaller electric field is applied, thereby surmounting similar methods. The particle enhances the effect of the electric field in its immediate vicinity, so reducing the field strength needed to achieve electroporation and thereby reducing the risk of damage to cells through high field exposure. Electroporation can be targeted to a subset of target cells by targeting the particles to surface markers on the target cell membrane.

GB Patent Application No. GB2495970A1 disclosing a targeted therapy associated nanoparticles with target cells (such as tumour cells) and exposes them to a time varying electric field sufficient to cause non-thermal electroporation of the cells. The electroporation may be reversible or irreversible depending on the strength of the applied field. The nanoparticles are conductive or high permittivity and enhance the electric field. They may be made from a metal or a metal oxide and are preferably iron oxide, gold, silver or platinum. The particles may have a coating containing antibodies, aptamers, or ligands which bind preferentially to receptors on the target cells. The coating may be uniform over the surface or it may be located on a specific region of the particle to promote binding in a preferred orientation. Electrodes may be located outside of the body or may be implanted or the method may be used in vitro.

U.S. Pat. No. 6,014,584A disclosing a method and apparatus for in vivo electroporation therapy. Using electroporation therapy (EPT) as described in the invention, tumors treated by a combination of electroporation using the apparatus of the invention and a chemotherapeutic agent caused regression of tumors in vivo. In one embodiment, the invention provides a method of EPT utilizing low voltage and long pulse length for inducing cell death. One embodiment of the invention includes a system for clinical electroporation that includes a needle array electrode having a “keying” element that determines the set point of the therapy voltage pulse and/or selectable array switching patterns. A number of electrode applicator designs permit access to and treatment of a variety of tissue sites. Another embodiment provides a laparoscopic needle applicator that is preferably combined with an endoscope for minimally invasive EPT.

U.S. Pat. No. 6,009,347A disclosing an electrode template apparatus, comprising a three dimensional support member having opposite surfaces, a plurality of bores extending through the support member and through the opposite surfaces, a plurality of conductors on the member separately connected to the plurality of bores, a plurality of electrodes selectively insertable in the plurality of bores so that each electrode is connected to at least one conductor for connecting the electrodes to a power supply.

U.S. Patent Application No. US2007025919AA disclosing methods and apparatus are provided for applying a fragment of a neurotoxin such as the active light chain (LC) of the botulinum toxin (BoNT), such as one of the serotypes A, B, C, D, E, F or G botulinum toxins, via permeabilization of targeted cell membranes to enable translocation of the botulinum neurotoxin light chain (BoNT-LC) molecule across the targeted cell membrane to the cell cytosol where a therapeutic response is produced in a mammalian system. The methods and apparatus include the use of catheter based delivery systems, non-invasive delivery systems, and transdermal delivery systems.

International Patent Application Publication No. WO9930655A1 disclosing systems and methods for selectively applying electrical energy to a target location within the head, and neck of a patient's body, particularly including tissue in the car, nose and throat. The present invention applies electrical energy to one or more electrode terminals in the presence of electrically conductive fluid to remove, and/or modify the structure of tissue structures. Depending on the specific procedure, the present invention may be used to volumetrically remove tissue (i.e., ablate or effect molecular dissociation of the tissue structure), shrink or contract collagen connective tissue, and/or coagulate severed blood vessels. For example, the present invention may be useful for ablation, and hemostasis of tissue in sinus surgery (e.g., chronic sinusitis or the removal of turbinates, polypectomies), collagen shrinkage, ablation, hemostasis in procedures for treating snoring, and obstructive sleep apnea (e.g., soft palate, such as the uvula, or tongue/pharynx stiffening, and midline glossectomies), for gross tissue removal, such as tonsillectomies, adenoidectomies, tracheal stenosis, vocal cord polyps, and lesions; or for the resection or ablation of facial tumors or tumor with the mouth, the pharynx such as glossectomies, laryngectomies, acoustic neuroma procedures, and nasal ablation procedures.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A method of reducing volume of a tissue without damaging epithelial tissue, the method comprising:
a. inserting an IRE device within a zone of a mouth or a nose;
b. enclosing an area of interest of said tissue within a space between at least two electrodes;
c. applying irreversible electroporation ablation treatment;

Example 2. The method according to example 1, wherein said irreversible electroporation ablation treatment comprises at least one ablation sequence comprising a frequency higher than 5 kHz.

Example 3. The method according to example 1 or example 2, wherein said applying at least one ablation sequence comprises providing at least one ablation sequence which provides irreversible electroporation ablation treatment with a reduction of function of the epithelial tissue below five percent.

Example 4. The method according to any one of examples 1-3, wherein said applying comprises non-thermally applying said at least one ablation sequence.

Example 5. The method according to any one of examples 1-4, further comprising stopping said irreversible electroporation ablation treatment before a reduction of function of the epithelial tissue reaches about five percent.

Example 6. The method according to any one of examples 1-5, further comprising applying a liquid or gel on said tissue for increasing contact and/or conduction.

Example 7. The method according to any one of examples 1-6, further comprising pulling away said area of interest of said tissue to separate said area of interest of said tissue from adjacent tissue.

Example 8. The method according to any one of examples 1-7, wherein said treatment is performed for a period of time of less than 90 seconds.

Example 9. The method according to any one of examples 1-8, wherein said treatment does not substantially affect tissues surrounding said area of interest to be treated.

Example 10. The method according to any one of examples 1-9, wherein said treatment is a non-invasive treatment.

Example 11. The method according to any one of examples 1-10, further comprising applying two or more sequences in said treatment.

Example 12. The method according to any one of examples 1-11, wherein said at least one sequence comprises the following parameters:

| | Sequence |
|---|---|
| Parameter | A |
| Voltage [V] | 0-400 |
| Frequency [kHz] | 20-500 |
| Positive Pulse width [μsec] | 1-20 |
| Negative Pulse width | 1-20 |
| Positive Pulse Amplitude | 0-400 |
| Negative Pulse Amplitude | 0-400 |
| Delay between pulses [μsec] | 0-8 |
| Number of Pulses in a burst | 0-60*10\hat{ }6 |
| Number of bursts | 0-100000 |
| Delay between bursts [ms] | 0-5000 |

Example 13. The method according to any one of examples 1-12, wherein said at least one sequence comprises the following parameters:

| | Sequence |
|---|---|
| Parameter | B |
| Voltage [V] | 100-2500 |
| Frequency [kHz] | 5-40 |
| Positive Pulse width [μsec] | 10-100 |
| Negative Pulse width | 10-100 |
| Positive Pulse Amplitude | 100-2400 |
| Negative Pulse Amplitude | 100-2400 |
| Delay between pulses [μsec] | 0-150 |
| Number of Pulses in a burst | 0-20*10\hat{ }6 |
| Number of bursts | 0-100000 |
| Delay between bursts [ms] | 0-1000 |

Example 14. The method according to any one of examples 1-13, wherein said at least one sequence comprises the following parameters:

| | Sequence |
|---|---|
| Parameter | C |
| Voltage [V] | 400-3000 |
| Frequency [kHz] | 10-500 |
| Positive Pulse width [μsec] | 1-5 |
| Negative Pulse width | 1-5 |
| Positive Pulse Amplitude | 400-3000 |
| Negative Pulse Amplitude | 400-3000 |
| Delay between pulses [μsec] | 0-50 |
| Number of Pulses in a burst | 0-40*10\hat{ }6 |
| Number of bursts | 0-100000 |
| Delay between bursts [ms] | 0-1000 |

Example 15. The method according to any one of examples 1-14, wherein said at least one sequence comprises a positive pulse width of from about 1 μsec to about 100 μsec.

Example 16. The method according to any one of examples 1-15, wherein said at least one sequence comprises a negative pulse width of from about 1 μsec to about 100 μsec.

Example 17. The method according to any one of examples 1-16, wherein said at least one sequence comprises a delay between pulses of from about 0 μsec to about 150 μsec.

Example 18. The method according to any one of examples 1-17, wherein said at least one sequence comprises a number of pulses in a burst of from about 5*10\hat{ }6 to about 60*10\hat{ }6.

Example 19. A device for performing irreversible electroporation ablation treatment to a subject, comprising:
a. a handle, comprising a first proximal end and a first distal end;
b. an elongated body comprising a second proximal end and a second distal end; said second proximal end being in mechanical communication with said first distal end of said handle;
c. an operational distal end comprising at least two electrodes separated by at least one groove, said groove sized and shaped to receive therein at least one tissue in need to receive said irreversible electroporation ablation treatment.

Example 20. The device according to example 19, wherein said at least one groove comprises a width of from about 1 mm and 15 mm and a height of from about 1 mm and 15 mm.

Example 21. The device according to example 20, further comprising a mechanism to modify said width of said groove.

Example 22. The device according to example 21, further comprising a mechanism to modify said height of said groove.

Example 23. The device according to any one of examples 19-22, wherein said at least two electrodes comprise a spherical form.

Example 24. The device according to any one of examples 19-23, wherein said at least two electrodes comprise an architecture that expands the emission of the electrical field to a wide area.

Example 25. The device according to any one of examples 19-24, wherein said at least two electrodes comprise an architecture that avoids concentrating the emission of the electrical to a single point in either electrode.

Example 26. The device according to any one of examples 19-25, wherein distance between the electrodes is presented as vector norm D=√(x\hat{ }2+y\hat{ }2) which is constant.

Example 27. The device according to any one of examples 19-26, further comprising at least one isolation material covering at least part of said operational distal end.

Example 28. The device according to any one of examples 19-27, wherein said elongated body comprise flexible parts.

Example 29. A device for performing irreversible electroporation ablation treatment to a subject, comprising:
a. a handle, comprising a first proximal end and a first distal end;
b. an elongated body comprising a second proximal end and a second distal end; said second proximal end being in mechanical communication with said first distal end of said handle;
c. an operational distal end comprising at least two arms and at least one space defined between said at least two arms; each of said at least two arms comprising at least one electrode; said at least one space sized and shaped to receive therein at least one tissue in need to receive said irreversible electroporation ablation treatment.

Example 30. The device according to example 29, wherein said space is modifiable by moving at least one of said at least two arms in relation to a pivot.

Example 31. The device according to example 29 or example 30, wherein said space is modifiable by linearly moving at least one of said at least two arms in relation to the other.

Example 32. The device according to any one of examples 29-31, wherein said at least one space comprises a width of from about 1 mm and 15 mm and a height of from about 1 mm and 15 mm.

Example 33. The device according to any one of examples 29-32, wherein said at least one electrode comprise a spherical form.

Example 34. The device according to any one of examples 29-33, wherein said at least one electrode comprise an architecture that expands the emission of the electrical field to a wide area.

Example 35. The device according to any one of examples 29-34, wherein said at least one electrode comprise an architecture that avoids concentrating the emission of the electrical to a single point in either electrode.

Example 36. The device according to any one of examples 29-35, wherein distance between the electrodes is presented as vector norm D=√(x\hat{ }2+y\hat{ }2) which is constant.

Example 37. The device according to any one of examples 29-36, further comprising at least one isolation material covering at least part of said operational distal end.

Example 38. The device according to any one of examples 29-37, wherein said elongated body comprise flexible parts.

Example 39. A method of IRE ablation treatment of a tissue, comprising:
a. grasping at least a part of said tissue within at least two elements if an IRE device;
b. reducing a distance between said at least two elements of said IRE device so as to compress said at least said part of said tissue grasped in said at least two elements;
c. applying irreversible electroporation ablation treatment according to said reduced distance between said at least two elements.

Example 40. The method according to example 39, wherein said tissue is one or more of tonsils, adenoid, base of the tongue and concha.

Example 41. The method according to example 39 or example 40, wherein at least one of said at least two elements of said IRE device comprises electrodes.

Example 42. The method according to any one of examples 39-41, wherein both of said at least two elements of said IRE device comprise electrodes.

Example 43. The method according to example 42, wherein said electrodes are positioned in a predetermined distance from each other.

Example 44. The method according to example 43, wherein said predetermined distance is compatible with preprogrammed IRE sequences and electric fields of said irreversible electroporation ablation treatment.

Example 45. The method according to any one of examples 39-49, further comprising reducing an electric field generated during said IRE ablation treatment by partially insulating said at least said part of said tissue from surrounding tissue.

Example 46. The method according to any one of examples 39-45, further comprising applying a liquid or gel on said tissue for increasing contact and/or conduction.

Example 47. The method according to any one of examples 39-46, further comprising pulling away said at least a part of said tissue to separate at least a part of said tissue from adjacent tissue.

Example 48. The method according to any one of examples 39-47, wherein said irreversible electroporation ablation treatment comprises at least one ablation sequence comprising a frequency higher than 5 kHz.

Example 49. The method according to any one of examples 39-48, wherein said applying comprises providing at least one ablation sequence which provides irreversible electroporation ablation treatment with a reduction of function of epithelial tissue below five percent.

Example 50. The method according to any one of examples 39-49, wherein said applying comprises non-thermally applying said irreversible electroporation ablation treatment.

Example 51. The method according to any one of examples 39-50, further comprising stopping said irreversible electroporation ablation treatment before a reduction of function of an epithelial tissue reaches about five percent.

Example 52. The method according to any one of examples 39-51, wherein said treatment is performed for a period of time of less than 90 seconds.

Example 53. The method according to any one of examples 39-52, wherein said treatment does not substantially affect tissues surrounding said at least a part of said tissue.

Example 54. The method according to any one of examples 39-53, wherein said treatment is a non-invasive treatment.

Example 55. The method according to any one of examples 39-54, further comprising applying two or more sequences in said treatment.

Example 56. The method according to example 48, wherein said at least one sequence comprises the following parameters:

| | Sequence |
|---|---|
| Parameter | A |
| Voltage [V] | 0-400 |
| Frequency [kHz] | 20-500 |
| Positive Pulse width [μsec] | 1-20 |
| Negative Pulse width | 1-20 |
| Positive Pulse Amplitude | 0-400 |
| Negative Pulse Amplitude | 0-400 |
| Delay between pulses [μsec] | 0-8 |
| Number of Pulses in a burst | 0-60*10\hat{ }6 |
| Number of bursts | 0-100000 |
| Delay between bursts [ms] | 0-5000 |

Example 57. The method according to example 48, wherein said at least one sequence comprises the following parameters:

| | Sequence |
|---|---|
| Parameter | B |
| Voltage [V] | 100-2500 |
| Frequency [kHz] | 5-40 |
| Positive Pulse width [μsec] | 10-100 |
| Negative Pulse width | 10-100 |
| Positive Pulse Amplitude | 100-2400 |
| Negative Pulse Amplitude | 100-2400 |
| Delay between pulses [μsec] | 0-150 |
| Number of Pulses in a burst | 0-20*10\hat{ }6 |
| Number of bursts | 0-100000 |
| Delay between bursts [ms] | 0-1000 |

Example 58. The method according to example 48, wherein said at least one sequence comprises the following parameters:

| | Sequence |
|---|---|
| Parameter | C |
| Voltage [V] | 400-3000 |
| Frequency [kHz] | 10-500 |
| Positive Pulse width [μsec] | 1-5 |
| Negative Pulse width | 1-5 |
| Positive Pulse Amplitude | 400-3000 |
| Negative Pulse Amplitude | 400-3000 |
| Delay between pulses [μsec] | 0-50 |
| Number of Pulses in a burst | 0-40*10\hat{ }6 |
| Number of bursts | 0-100000 |
| Delay between bursts [ms] | 0-1000 |

Example 59. The method according to example 48, wherein said at least one sequence comprises a positive pulse width of from about 1 μsec to about 100 μsec.

Example 60. The method according to example 48, wherein said at least one sequence comprises a negative pulse width of from about 1 μsec to about 100 μsec.

Example 61. The method according to example 48, wherein said at least one sequence comprises a delay between pulses of from about 0 μsec to about 150 μsec.

Example 62. The method according to example 48, wherein said at least one sequence comprises a number of pulses in a burst of from about 5*10\hat{ }6 to about 60*10\hat{ }6.

Example 63. A method of IRE ablation treatment of a tissue, comprising:
a. providing a IRE ablation device comprising electrodes located at a fixed distance between each other and a space defined between said electrodes;
b. positioning said device onto said tissue so as to allocate said tissue within said space and between said electrodes.

Example 64. The method according to example 63, wherein said tissue is one or more of tonsils, adenoid, base of the tongue and concha.

Example 65. The method according to example 63 or example 64, wherein said electrodes are positioned in a predetermined distance from each other.

Example 66. The method according to example 65, wherein said predetermined distance is compatible with preprogrammed IRE sequences and electric fields of said IRE ablation treatment.

Example 67. The method according to any one of examples 63-66, further comprising reducing an electric field generated during said IRE ablation treatment by partially insulating at least part of said tissue from surrounding tissue.

Example 68. The method according to any one of examples 63-67, further comprising applying a liquid or gel on said tissue for increasing contact and/or conduction.

Example 69. The method according to any one of examples 63-68, further comprising pulling away an area of interest of said tissue to separate said area of interest of said tissue from adjacent tissue.

Example 70. The method according to any one of examples 63-69, wherein said IRE ablation treatment comprises at least one ablation sequence comprising a frequency higher than 5 kHz.

Example 71. The method according to example 70, wherein said at least one ablation sequence comprises providing at least one ablation sequence which provides irreversible electroporation ablation treatment with a reduction of function of epithelial tissue below five percent.

Example 72. The method according to any one of examples 63-71, wherein said IRE ablation treatment comprises non-thermally applying said IRE ablation treatment comprises.

Example 73. The method according to any one of examples 63-72, further comprising stopping said IRE ablation treatment before a reduction of function of an epithelial tissue reaches about five percent.

Example 74. The method according to any one of examples 63-73, wherein said treatment is performed for a period of time of less than 90 seconds.

Example 75. The method according to any one of examples 63-74, wherein said treatment does not substantially affect tissues surrounding said tissue to be treated.

Example 76. The method according to any one of examples 63-75, wherein said treatment is a non-invasive treatment.

Example 77. The method according to any one of examples 63-76, further comprising applying two or more sequences in said treatment.

Example 78. The method according to example 70, wherein said at least one sequence comprises the following parameters:

| | Sequence |
|---|---|
| Parameter | A |
| Voltage [V] | 0-400 |
| Frequency [kHz] | 20-500 |
| Positive Pulse width [μsec] | 1-20 |
| Negative Pulse width | 1-20 |
| Positive Pulse Amplitude | 0-400 |
| Negative Pulse Amplitude | 0-400 |
| Delay between pulses [μsec] | 0-8 |
| Number of Pulses in a burst | 0-60*10\hat{ }6 |
| Number of bursts | 0-100000 |
| Delay between bursts [ms] | 0-5000 |

Example 79. The method according to example 70, wherein said at least one sequence comprises the following parameters:

| | Sequence |
|---|---|
| Parameter | B |
| Voltage [V] | 100-2500 |
| Frequency [kHz] | 5-40 |
| Positive Pulse width [μsec] | 10-100 |
| Negative Pulse width | 10-100 |
| Positive Pulse Amplitude | 100-2400 |
| Negative Pulse Amplitude | 100-2400 |
| Delay between pulses [μsec] | 0-150 |
| Number of Pulses in a burst | 0-20*10\hat{ }6 |
| Number of bursts | 0-100000 |
| Delay between bursts [ms] | 0-1000 |

Example 80. The method according to example 70, wherein said at least one sequence comprises the following parameters:

| | Sequence |
|---|---|
| Parameter | C |
| Voltage [V] | 400-3000 |
| Frequency [kHz] | 10-500 |
| Positive Pulse width [μsec] | 1-5 |
| Negative Pulse width | 1-5 |
| Positive Pulse Amplitude | 400-3000 |
| Negative Pulse Amplitude | 400-3000 |
| Delay between pulses [μsec] | 0-50 |
| Number of Pulses in a burst | 0-40*10\hat{ }6 |
| Number of bursts | 0-100000 |
| Delay between bursts [ms] | 0-1000 |

Example 81. The method according to example 70, wherein said at least one sequence comprises a positive pulse width of from about 1 μsec to about 100 μsec.

Example 82. The method according to example 70, wherein said at least one sequence comprises a negative pulse width of from about 1 μsec to about 100 μsec.

Example 83. The method according to example 70, wherein said at least one sequence comprises a delay between pulses of from about 0 μsec to about 150 μsec.

Example 84. The method according to example 70, wherein said at least one sequence comprises a number of pulses in a burst of from about 5*10\hat{ }6 to about 60*10\hat{ }6.

Example 85. A device for performing irreversible electroporation ablation treatment to a subject, comprising:
a. a handle, comprising a first proximal end and a first distal end;
b. an elongated body comprising a second proximal end and a second distal end; said second proximal end being in mechanical communication with said first distal end of said handle;
c. an operational distal end comprising:
   i. a distal arm located and connected at the most distal end of said elongated body;
   ii. a proximal arm located proximally to said distal arm and connected to said elongated body;
   iii. a middle element, located at a space defined between said distal and said proximal arm; said middle element connected to said elongated body and comprising electrodes on at least one surface; said space sized and shaped to receive therein at least one tissue in need to receive said irreversible electroporation ablation treatment.

Example 86. The device according to example 85, further comprising at least one isolation material covering at least part of said operational distal end.

Example 87. The device according to example 85 or example 86, wherein said elongated body comprise flexible parts.

According to an aspect of some embodiments of the present invention there is provided a method of providing irreversible electroporation ablation treatment to a subject, comprising:
a. enclosing an area of interest to be treated in said subject with at least one electrode;
b. applying at least one ablation sequence comprising a frequency higher than 5 kHz.

According to some embodiments of the invention, said treatment is performed for a period of time from about 20 seconds to about 90 seconds.

According to some embodiments of the invention, the whole procedure is performed for a period of time from about 20 seconds to about 90 seconds, while the at least one ablation sequence is performed for a period of time from about 0.1 seconds to about 20 seconds.

According to some embodiments of the invention, said treatment does not affect tissues surrounding said area of interest to be treated.

According to some embodiments of the invention, said treatment is a non-invasive treatment.

According to some embodiments of the invention, further comprising using at least two electrodes.

According to some embodiments of the invention, a distance between said at least two electrodes is from about 5% to about 60% higher in comparison to known IRE techniques.

According to some embodiments of the invention, a distance between said at least two electrodes is from about 0.5 cm to about 3 cm.

According to some embodiments of the invention, a distance between said at least two electrodes is from about 0.01 mm to about 30 mm.

According to some embodiments of the invention, further comprising applying two or more sequences in said treatment.

According to some embodiments of the invention, said at least one sequence comprises the following parameters:

| | Sequence |
|---|---|
| Parameter | A |
| Voltage [V] | 0-400 |
| Frequency [kHz] | 20-500 |
| Positive Pulse width [μsec] | 1-20 |
| Negative Pulse width | 1-20 |
| Positive Pulse Amplitude | 0-400 |
| Negative Pulse Amplitude | 0-400 |
| Delay between pulses [μsec] | 0-8 |
| Number of Pulses in a burst | 0-60*10\hat{ }6 |
| Number of bursts | 0-100000 |
| Delay between bursts [ms] | 0-5000 |

According to some embodiments of the invention, said at least one sequence comprises the following parameters:

| | Sequence |
|---|---|
| Parameter | B |
| Voltage [V] | 100-2500 |
| Frequency [kHz] | 5-40 |
| Positive Pulse width [μsec] | 10-100 |
| Negative Pulse width | 10-100 |
| Positive Pulse Amplitude | 100-2400 |
| Negative Pulse Amplitude | 100-2400 |
| Delay between pulses [μsec] | 0-150 |
| Number of Pulses in a burst | 0-20*10\hat{ }6 |
| Number of bursts | 0-100000 |
| Delay between bursts [ms] | 0-1000 |

According to some embodiments of the invention, said at least one sequence comprises the following parameters:

| | Sequence |
|---|---|
| Parameter | C |
| Voltage [V] | 400-3000 |
| Frequency [kHz] | 10-500 |
| Positive Pulse width [μsec] | 1-5 |
| Negative Pulse width | 1-5 |
| Positive Pulse Amplitude | 400-3000 |
| Negative Pulse Amplitude | 400-3000 |
| Delay between pulses [μsec] | 0-50 |
| Number of Pulses in a burst | 0-40*10\hat{ }6 |
| Number of bursts | 0-100000 |
| Delay between bursts [ms] | 0-1000 |

According to some embodiments of the invention, said at least one sequence comprises a voltage of from about 400V to about 3000V.

According to some embodiments of the invention, said at least one sequence comprises a frequency of from about 5 kHz to about 500 kHz.

According to some embodiments of the invention, said at least one sequence comprises a positive pulse width of from about 1 μsec to about 100 μsec.

According to some embodiments of the invention, said at least one sequence comprises a negative pulse width of from about 1 μsec to about 100 μsec.

According to some embodiments of the invention, said at least one sequence comprises a positive pulse amplitude of from about 400V to about 3000V.

According to some embodiments of the invention, said at least one sequence comprises a negative pulse amplitude of from about 400 to about 3000.

According to some embodiments of the invention, said at least one sequence comprises a delay between pulses of from about 0 μsec to about 150 μsec.

According to some embodiments of the invention, said at least one sequence comprises a number of pulses in a burst of from about 5*10\hat{ }6 to about 60*10\hat{ }6.

According to some embodiments of the invention, said at least one sequence comprises a number of bursts of from about 1 to about 100000.

According to some embodiments of the invention, said at least one sequence comprises a delay between bursts of from about 0.1 ms to about 5000 ms.

According to some embodiments of the invention, said at least one sequence comprises a delay between bursts of from about 0.01 ms to about 5000 ms.

According to an aspect of some embodiments of the present invention there is provided a device for performing irreversible electroporation ablation treatment to a subject, comprising:
a. a handle, comprising a proximal end and a distal end;
b. an element having a concave shape located at said distal end of said handle;
c. at least one electrode configured to electroporation ablation energy, located within said concave shape of said element;
d. an isolation material covering the external surface of said element.

According to some embodiments of the invention, two devices are used for performing said irreversible electroporation ablation treatment.

According to some embodiments of the invention, said device is used in concomitance with a needle for performing said irreversible electroporation ablation treatment.

According to some embodiments of the invention, said concave shape of said element comprises two electrodes.

According to some embodiments of the invention, further comprising an opening within said concave shape of said element for suction.

According to some embodiments of the invention, said handles comprise flexible parts.

According to some embodiments of the invention, a second electrode is used outside the body of said subject but in contact with at least one part of said body of said subject.

According to some embodiments of the invention, said element is atraumatic.

According to some embodiments of the invention, further comprising hardware for monitoring a heart of said subject during said treatment.

According to an aspect of some embodiments of the present invention there is provided a method of performing irreversible electroporation ablation treatment to at least one tonsil of a subject, comprising:
a. enclosing said at least one tonsil (and/or adenoid and/or base of tongue) from a first side with a first electrode;
b. enclosing said at least one tonsil from a second side with a second electrode;
c. applying at least one ablation sequence.

According to some embodiments of the invention, further comprising slightly pulling said at least one tonsil once enclosed from both sides to separate said at least one tonsil from adjacent tissue.

According to some embodiments of the invention, said first electrode and said second electrode are according to the device as disclosed above.

According to some embodiments of the invention, said applying at least one ablation sequence is according to the method as disclosed above.

According to an aspect of some embodiments of the present invention there is provided a method of performing irreversible electroporation ablation treatment to an inferior turbinate of a subject, comprising:
a. bringing a first electrode in contact with at least one first portion of said inferior turbinate;
b. bringing a second electrode in contact with at least one-second portion of said inferior turbinate;
c. applying at least one ablation sequence.

According to some embodiments of the invention, said first electrode and said second electrode are according to the device as disclosed above.

According to some embodiments of the invention, said first electrode and/or said second electrode are needless.

According to some embodiments of the invention, said applying at least one ablation sequence is according to the method as disclosed above.

According to an aspect of some embodiments of the present invention there is provided a method of performing irreversible electroporation ablation treatment to a prostate of a subject, comprising:
a. bringing a first electrode in contact with at least one portion of said prostate;
b. bringing a second electrode in contact with at least one location on the outside of a body of said subject; said at least one location being at a distance of said first electrode of from about 0.5 cm to about 5 cm; optionally said at least one location being at a distance of said first electrode of from about 0.01 mm to about 5 cm;
c. applying at least one ablation sequence.

According to some embodiments of the invention, said first electrode and said second electrode are according to the device as disclosed above.

According to some embodiments of the invention, said first electrode and/or said second electrode are needles.

According to some embodiments of the invention, said applying at least one ablation sequence is according to the method as disclosed above.

According to an aspect of some embodiments of the present invention there is provided a method of performing irreversible electroporation ablation treatment to at least one location at the base of the tongue of a subject, comprising:
a. enclosing said at least one location at the base of the tongue from a first side with a first electrode;
b. enclosing said at least one location at the base of the tongue from a second side with a second electrode;
c. applying at least one ablation sequence.

According to some embodiments of the invention, further comprising slightly pulling said at least one location at the base of the tongue once enclosed from both sides to separate said at least one location at the base of the tongue from adjacent tissue.

According to some embodiments of the invention, said first electrode and said second electrode are according to the device as disclosed above.

According to some embodiments of the invention, said applying at least one ablation sequence is according to the method as disclosed above.

According to an aspect of some embodiments of the present invention there is provided a method of performing irreversible electroporation ablation treatment to at least one adenoid of a subject, comprising:
a. enclosing said at least one adenoid from a first side with a first electrode;
b. enclosing said at least one adenoid from a second side with a second electrode;
c. applying at least one ablation sequence.

According to some embodiments of the invention, further comprising slightly pulling said at least one adenoid once enclosed from both sides to separate said at least one adenoid from adjacent tissue.

According to some embodiments of the invention, said first electrode and said second electrode are according to the device as disclosed above.

According to some embodiments of the invention, said applying at least one ablation sequence is according to the method as disclosed above.

According to an aspect of some embodiments of the present invention there is provided a method of performing irreversible electroporation ablation treatment to at least one concha of a subject, comprising:
a. enclosing said at least one concha from a first side with a first electrode;
b. enclosing said at least one concha from a second side with a second electrode;
c. applying at least one ablation sequence.

According to some embodiments of the invention, further comprising slightly pulling said at least one concha once enclosed from both sides to separate said at least one concha from adjacent tissue.

According to some embodiments of the invention, said first electrode and said second electrode are according to the device as disclosed above.

According to some embodiments of the invention, said applying at least one ablation sequence is according to the method as disclosed above.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1a-b are flowcharts of exemplary general treatment methods, according to some embodiments of the invention;

FIG. 2 is a graph of an exemplary ablation sequence, according to some embodiments of the invention;

FIG. 3 is a graph of an exemplary ablation sequence, according to some embodiments of the invention;

FIG. 4 is a graph of an exemplary ablation sequence, according to some embodiments of the invention;

FIG. 5 is a graph of an exemplary ablation sequence, according to some embodiments of the invention;

FIGS. 6a-6b are schematic representation of exemplary electroporation cups, according to some embodiments of the invention;

FIG. 7 is a schematic representation of electrode forceps, according to some embodiments of the invention;

FIG. 8 is a schematic representation of an electroporation treatment for the tonsils utilizing forceps, according to some embodiments of the invention;

FIG. 9 is a schematic representation of an electroporation treatment for the tonsils utilizing a cup electrode, according to some embodiments of the invention;

FIG. 10 is a schematic representation of an isolation and electroporation treatment for the tonsils utilizing a cup electrode, according to some embodiments of the invention;

FIG. 11 is a schematic representation of an isolation and electroporation treatment for the tonsils utilizing a cup electrode and a needle, according to some embodiments of the invention;

FIGS. 12a-d are schematic representations of an electroporation treatment for the inferior turbinate/adenoids utilizing a probe or a needle, according to some embodiments of the invention;

FIG. 13 is a schematic representation of a prostate treatment, according to some embodiments of the invention;

FIG. 14 is a schematic representation of a prostate treatment, according to some embodiments of the invention;

FIGS. 15a-f are schematic representations of exemplary treatment of the tonsils, according to some embodiments of the invention;

FIGS. 16a-g are schematic representations of exemplary IRE devices, according to some embodiments of the invention;

FIGS. 17a-i are schematic representations of exemplary IRE devices, according to some embodiments of the invention;

FIGS. 18a-c are schematic representations of exemplary IRE devices, according to some embodiments of the invention;

FIGS. 19a-f are schematic representations of exemplary mechanism of actions of exemplary IRE devices, according to some embodiments of the invention; and

FIG. 20 is a schematic representation of an exemplary electrode configuration of the IRE system, according to some embodiments of the invention.

### DESCRIPTION OF SPECIFIC EMBODIMENTS OF THE INVENTION

The present invention, in some embodiments thereof, relates to electroporation treatments and, more particularly, but not exclusively, to non-invasive electroporation treatments.

### Overview

An aspect of some embodiments of the invention relates to IRE treatments to one or more tissues. In some embodiments, the whole duration of the treatment is shorter than gold standard procedures, for example IRE procedure for tonsil mass reduction will shorter the gold standard tonsillectomy/tonsillectomy, for example, such IRE procedure treatment will take under 10 minutes. In some embodiments, the treatment does not substantially damage tissues around the zone of interest, for example, no damage to the outer mucosa tissue, while treating the inner lymphatic tissue. In some embodiments, the treatment causes minimal damage to the tissues around the zone of interest. In some embodiments, the IRE treatments comprise the use of high frequencies. In some embodiments, the treatment is a non-invasive treatment. In some embodiments, recuperation time after the treatment is short due to the non-invasiveness nature of the treatment. In some embodiments, the treatment does not cause edemas or inflammations or bleeding, thus reducing pain and preventing unnecessary scarring and swelling.

An aspect of some embodiments of the invention relates to IRE treatments to one or more tissues. In some embodiments, the whole duration of the treatment is very short, for example between about 20 seconds to about 90 seconds. In some embodiments, the treatment does not damage tissues around the zone of interest. In some embodiments, the treatment causes minimal damage to the tissues around the zone of interest. In some embodiments, the IRE treatments comprise the use of high frequencies. In some embodiments, the treatment is a non-invasive treatment. In some embodiments, recuperation time after the treatment is very short due to the non-invasiveness nature of the treatment. In some embodiments, the treatment does not cause edemas or inflammations, thus reducing pain and preventing unnecessary scarring and swelling.

An aspect of some embodiments of the invention relates to non-thermal IRE treatments to one or more tissues and an optionally is a selective mode where in some cases only target tissue is ablated while surrounding tissues remain intact. In some embodiments, the whole duration of the treatment is very short, for example between about 2 seconds to about 90 seconds. In some embodiments, the treatment does not damage tissues around the zone of interest. In some embodiments, the treatment causes minimal damage to the tissues around the zone of interest. In some embodiments, the IRE treatments comprise the use of high frequencies. In some embodiments, the treatment is a non-invasive treatment. In some embodiments, recuperation time after the treatment is very short due to the non-invasiveness nature of the treatment. In some embodiments, the treatment does not cause necrosis, edemas, or inflammations, thus reducing pain and preventing unnecessary scarring, and swelling.

An aspect of some embodiments of the invention related to specific frequency sequences for IRE. In some embodiments, the frequency sequences for IRE allow greater distancing of electrodes from one another in comparison to known IRE techniques. In some embodiments, the frequency sequences for IRE allow from about 15% to about 20% greater distancing of electrodes from one another in comparison to known IRE techniques. In some embodiments, the frequency sequences for IRE allow from about 10% to about 40% greater distancing of electrodes from one another in comparison to known IRE techniques. In some embodiments, the frequency sequences for IRE allow from about 5% to about 60% greater distancing of electrodes from one another in comparison to known IRE techniques. In some embodiments, the distance between the two electrodes is very short, for example from about 1 cm to about 2 cm, optionally from about 0.5 cm to about 3 cm, optionally from about 0.25 cm to about 5 cm. In some embodiments, the distance between the two electrodes is very short, for example from about 0.01 mm to about 2 cm, optionally from about 0.5 cm to about 3 cm, optionally from about 0.001 cm to about 5 cm. In some embodiments, the frequencies and the frequency sequences do not cause nerve or epithelium layer damage during the treatment. In some embodiments, the frequencies and the frequency sequences used during the treatment cause minimal nerve damage. In some embodiments, higher frequencies are used to cause less responses from the nerves, which can potentially reduce muscle contractions. In some embodiments, the frequencies are not high enough as to be unspecific. In some embodiments, the use of IRE damages the cell membrane with reduced or non-existent heat damage or cardiac effect. In some embodiments, a potential advantage is that it potentially facilitates the recovery time after the procedure. In some embodiments, the fact that there is no thermal damage and no nerve damage or immediate damage to the treated tissue, potentially reduces pain during the recovery from the procedure, as well as potentially avoid other heat-induced symptoms like infections, coagulation and swellings that get significant when using the airways. In some embodiment limited heat damage is induced as part of the treatment i.e., heat nerve damage.

An aspect of some embodiments of the invention relates to the combination between dedicated hardware and specific activation protocols of the hardware for IRE treatments.

An aspect of some embodiments of the invention relates to encapsulating tissues for the administration of IRE treatments. In some embodiments, the encapsulation of the tissue comprises the use of one or more tools which encapsulate the tissue and potentially isolate the encapsulated tissue from the adjacent tissue, thereby potentially isolating the tissue that requires the treatment without providing treatment to those adjacent tissues. In some embodiments, encapsulating a tissue comprises the use of two concave tools, which together form an isolation chamber. In some embodiments, when the tool(s) encapsulate the tissue, the part of the tissue that still connects the encapsulated tissue with the adjacent tissue, is subjected to mechanical deformations that potentially stop the transmission of the IRE treatment to adjacent tissues. In some embodiments, encapsulating of tissues is performed especially, but not limited to tissues in the mouth and in the nose. In some embodiments, the tools are covered with one or more materials that can potentially provide better conduction of energy for the IRE treatment, or materials that numb the tissue being treated and possibly the adjacent tissue as well.

An aspect of some embodiments of the invention relates to selectively non-thermal ablating tissue utilizing irreversible electroporation (IRE) techniques/treatments. In some embodiments, the affected tissue is one or more of fibrous tissue, muscle, and lymphoid tissues. In some embodiments, the treatment does not damage the epithelium and/or the squamous epithelium and/or the respiratory epithelium, and/or non-keratinizing squamous epithelium, and/or mucous membranes. In some embodiments, the IRE treatment is configured to reduce tissue volume without damaging the epithelium. In some embodiments, the IRE treatment is configured to reduce tissue volume with a reduction of function of the epithelial tissue below five percent (reduction of function=<5%). In some embodiments, IRE treatments are used in one or more of the throat and the nose. In some embodiments, IRE treatments are used for a selective tonsillectomy or tonsillectomy, or adenoidectomy. In some embodiments, the selective ablation is performed as to affect the tissue at different distribution of treatment and at different depths. In some embodiments, the distribution of treatment is a controlled distribution, meaning that the distribution of the treatment is known in advance and according to the treatment parameters. In some embodiments, the depth of treatment is a controlled depth, meaning that the depth of the treatment is known in advance and according to the treatment parameters. In some embodiments, the IRE treatment causes focal necrosis and/or lymphocytic infiltration with some macrophages, while leaving epithelial structures are intact. In some embodiments, the pulse characteristics delivered by the device are configured to ablate tissues (for example lymphoid follicles, fibrous tissue and muscle cells) in various depths while keeping or at least creating minimal damage to the squamous epithelium of the location of treatment (for example: the tonsils, adenoid, concha or base of tongue). In some embodiments, the IRE device is configured to provide optimized treatment because of a combination of operating characteristics as a function of one or more of the following parameters: distance between the electrodes, electrodes diameter, electrode electric potential, pulse duration and number of pulses per single treatment. In some embodiments, the distance between the electrodes is from about 0 mm to about 15 mm. In some embodiments, the diameter of the electrodes is from about 1 mm to about 8 mm. In some embodiments, the electrodes potential is from about 1,000V to about 1,5000V. In some embodiments, the pulse duration is from about 1 microsecond and about 5 microseconds. In some embodiments, the sequence of pulses comprises from about 6 pulses to about 16 pulses. In some embodiments, a combination of a set of pulses comprises a delay between pulses of from about 1 ms delay to about 50 ms delay.

In some embodiments, during treatment, the impedance between electrodes indicates the treated tissue conductivity, which varies between about 80 ohm and about 120 ohm. In some embodiments, the user is notified by the system using for example an audio, audio-visual or visual indication when the impedance range is not within the anticipated range. In some embodiments, in such cases, the user may improve the impedance measurements by improving the contact between the electrodes and the treated tissue.

Before explaining at least one embodiment of the invention in detail, it is to be understood that the invention is not necessarily limited in its application to the details of construction and the arrangement of the components and/or methods and/or exemplified by the Examples set forth in the following description and/or illustrated in the drawings. The invention is capable of other embodiments or of being practiced or carried out in various ways.

### Exemplary General Method of the Treatment

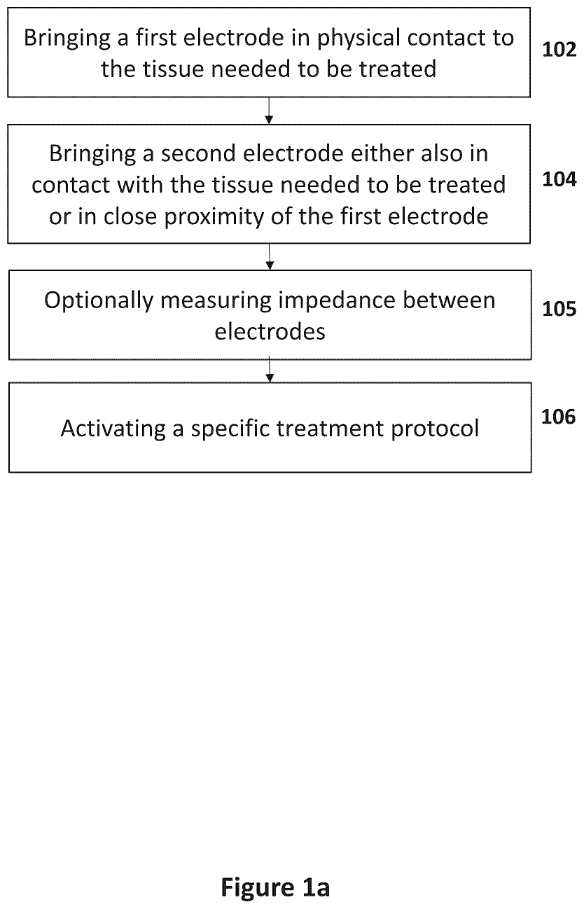

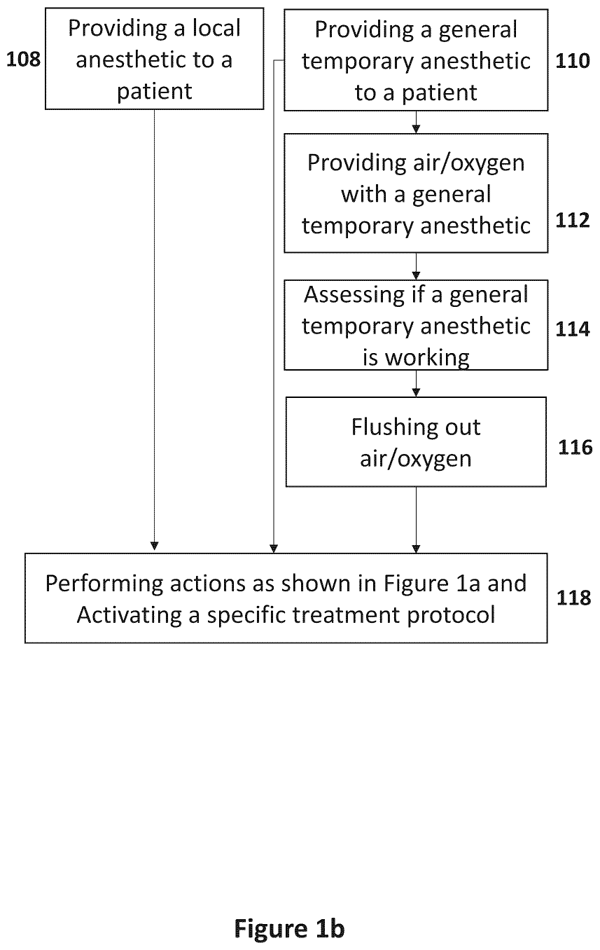

Referring now to FIG. 1a, showing a flowchart of an exemplary general treatment method, according to some embodiments of the invention. In some embodiments, an exemplary treatment method comprises:

1. Bringing a first electrode in physical contact to the tissue needed to be treated 102;

2. Bringing at least one second electrode (in some embodiments it is possible to use more than two) either also in contact with the tissue needed to be treated or in close proximity of the first electrode 104. In some embodiments, optionally, the user measures the impedance between electrodes to assess the correct functioning of the device and that the correct settings are achieved for the IRE treatment 105. In some embodiments, the maximal distance between electrodes, when one of the electrodes is not in direct contact with the tissue to be treated is up to 20 cm. Optionally from about 10 cm to about 20 cm. Optionally from about 7 cm to about 30 cm. Optionally from about 5 cm to about 40 cm. In some embodiments, the distance between the two electrodes is very short, for example from about 1 cm to about 2 cm, optionally from about 0.5 cm to about 3 cm, optionally from about 0.25 cm to about 5 cm.

In some embodiments, at least one electrode will be used, and another surface will absorb the electric current on the other side of the tissue.

3. Activating a specific treatment protocol 106 (see below Exemplary Frequency Sequences for IRE treatment).

In some embodiments, the treatment is performed without intubation of the patient. In some embodiments, the treatment is performed using a local anesthetic. In some embodiments, the treatment is performed either with or without the administration of a muscle relaxant. In some embodiments, during the treatment, the area of treatment is optionally cooled, in order to potentially avoid heat to damage the tissues in the vicinity of the treatment. In some embodiments, the user utilizes an endoscope during the procedure to visualize the treatment areas. In some embodiments, one of the two electrodes is not in direct contact with the relevant tissue to be treated in the patient. In some embodiments, the treatment is performed after the administration of anesthesia. In some embodiments, anesthesia is provided using a mask. In some embodiments, oxygen is further provided with the mask. In some embodiments, the treatment is provided via the mask and while the mask is still mounted on the patient face. In some embodiments, oxygen is not delivered via the mask during the specific time of activation of the IRE device.

Referring now to FIG. 1b, showing a flowchart of an exemplary general treatment method, according to some embodiments of the invention. In some embodiments, an exemplary treatment method comprises:

Providing a type of local anesthetic to the patient 108. In some embodiments, the local anesthetic is provided using a syringe. In some embodiments, the local anesthetic is provided using a spray.

In some embodiments, instead of providing a local anesthetic, the operator provides a general anesthetic to the patient 110. In some embodiments, the general anesthetic is provided for a short period of time, for example, for from about 30 seconds to about 3 minutes. Optionally, from about 15 seconds to about 5 minutes. In some embodiments, the general anesthetic is provided so as to anesthetize the patient for a very short time, for example, for about 0.5 seconds, 1 second, 5 seconds, 30 seconds or 60 seconds. In some embodiments, the time of action required for the anesthetic is the time required to provide the IRE treatment and no more. For example, if the IRE treatment takes 1 second, then the general anesthetic is going to be provided so as to last about 1 second or more. In some embodiments, the general temporary anesthetic is provided without any kind of intubation and the operator performs the actions as disclosed in FIG. 1a until activating the specific treatment protocol 118. In some embodiments, the general anesthetics provided in concomitance with oxygen 112, as usually done in these cases. In some embodiments, the operator then assesses if the general anesthetic is working 114. In some embodiments, when the answer is no, the operator will wait until the general anesthetic will take effect. In some embodiments, when the answer is yes, then the user will flush out excess of oxygen from the patient 116. In some embodiments, flushing out excess oxygen is done by flushing in regular air. In some embodiments, flushing out excess oxygen is performed as a preventive measure to avoid causing damage to the patient due to the excess of oxygen and the IRE treatment, which can cause for example burns to the patient. In some embodiments, after flushing the excess of oxygen the operator performs the actions as disclosed in FIG. 1a until activating the specific treatment protocol 118. In some embodiments, after providing the local anesthetic 108, the operator performs the actions as disclosed in FIG. 1a until activating the specific treatment protocol 118.

In some embodiments, the treatment does not damage the epithelium and/or the squamous epithelium and/or the respiratory epithelium. In some embodiments, the IRE treatment is configured to reduce tissue volume without damaging the epithelium. In some embodiments, the IRE treatment is configured to reduce tissue volume with a reduction of function of the epithelial tissue below five percent (reduction of function=<5%).

### Exemplary Frequency Sequences for IRE Treatment

In some embodiments, IRE treatments are characterized by a combination of one or more frequency sequences. In some embodiments, each sequence is characterized by one or more of the following parameters: Voltage [V], Frequency [kHz], Positive Pulse width [μsec], Negative Pulse width [μsec], Positive Pulse Amplitude, Negative Pulse Amplitude, Delay between pulses [μsec], Number of pulses in a burst, Number of bursts, Delay between bursts [milliseconds (ms)].

In some embodiments, exemplary frequency sequences used in IRE treatments are as follows:

| | Sequence | | |
|---|---|---|---|
| Parameter | A | B | C |
| Voltage [V] | 0-400 | 100-2500 | 400-3000 |
| Frequency [kHz] | 20-500 | 5-40 | 10-500 |
| Positive Pulse width [μsec] | 1-20 | 10-100 | 1-5 |
| Negative Pulse width [μsec] | 1-20 | 10-100 | 1-5 |
| Positive Pulse Amplitude | 0-400 | 100-2400 | 400-3000 |
| Negative Pulse Amplitude | 0-400 | 100-2400 | 400-3000 |
| Delay between pulses [μsec] | 0-8 | 0-150 | 0-50 |
| Number of Pulses in a burst | 0-60*10\hat{ }6 | 0-20*10\hat{ }6 | 0-40*10\hat{ }6 |
| Number of bursts | 0-100000 | 0-100000 | 0-100000 |
| Delay between bursts [ms] | 0-5000 | 0-1000 | 0-1000 |

In some embodiments, the combination of the one or more frequency sequences are evaluated and/or calculated so as to potentially avoid damage to the epithelium and/or the squamous epithelium and/or the respiratory epithelium. In some embodiments, the combination of the one or more frequency sequences are evaluated and/or calculated so as to reduce tissue volume without damaging the epithelium. In some embodiments, the combination of the one or more frequency sequences are evaluated and/or calculated so as to reduce tissue volume with a reduction of function of the epithelial tissue below five percent (reduction of function=<5%).

In some embodiments, ablation treatments comprise one or more of the abovementioned sequences. In order to enable a person having skills in the art to understand the invention, the following examples will be provided. It should be understood that those examples should not be limiting in any way, and other sequences can be used and are intended to be in the scope of the invention.

### Example 1

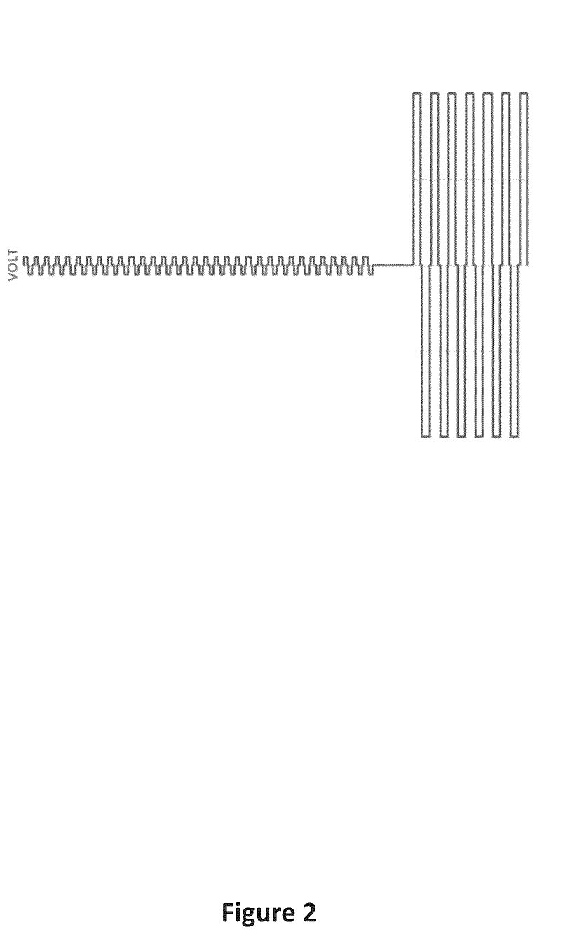

In some embodiments, ablation treatments begin with a 10 second ablation of sequence A followed by 100 microseconds of burst of sequence B, as can be seen for example in FIG. 2 and as following:

Sequence

| Parameter | A | B |
|---|---|---|
| Voltage [V] | 50 | 2000 |
| Frequency [kHz] | 500 | 40 |
| Positive Pulse width [μsec] | 1 | 12.5 |
| Negative Pulse width | 1 | 12.5 |
| Positive Pulse Amplitude | 50 | 2000 |
| Negative Pulse Amplitude | 50 | 2000 |
| Delay between pulses [μsec] | 0 | 2 |
| Number of Pulses in a burst | 10*10\hat{ }6 | 4*10\hat{ }6 |
| Number of bursts | 1 | 10 |
| Delay between bursts [ms] | 0 | 10 |

### Example 2

In some embodiments, ablation treatments comprise a combination of sequences with interleaved pulses, as following:

Sequence

| Parameter | B | C |
|---|---|---|
| Voltage [V] | 200 | 1200 |
| Frequency [kHz] | 35 | 35 |
| Positive Pulse width [μsec] | 12 | 2 |
| Negative Pulse width | 12 | 2 |
| Positive Pulse Amplitude | 200 | 1200 |
| Negative Pulse Amplitude | 200 | 1200 |
| Delay between pulses [μsec] | 2.5 | 12.5 |
| Number of Pulses in a burst | 10*10\hat{ }6 | 10*10\hat{ }6 |
| Number of bursts | 10 | 10 |
| Delay between bursts [ms] | 10 | 10 |

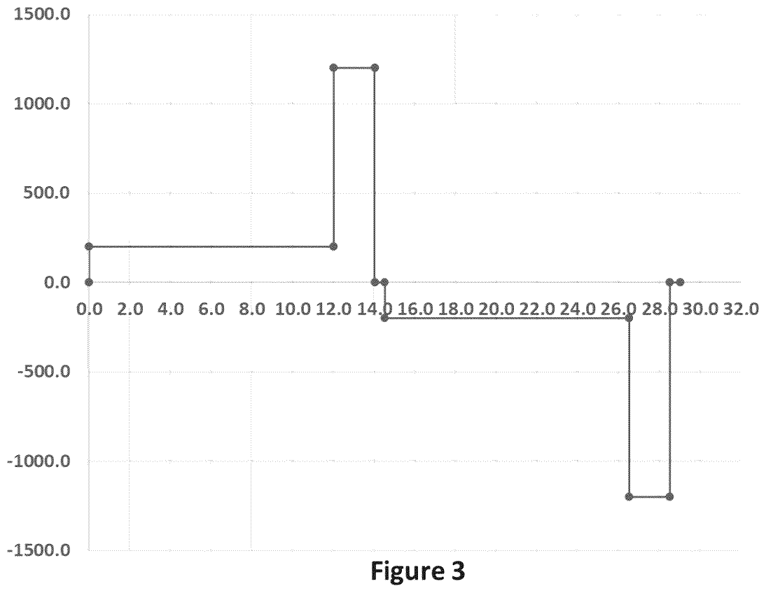

FIG. 3 show an exemplary single pulse. In some embodiments, the single pulse is repeated 10 times in a burst, and 10 bursts are repeated with a 10 milliseconds delay between bursts—providing a total ablation time of about 100 milliseconds.

### Example 3

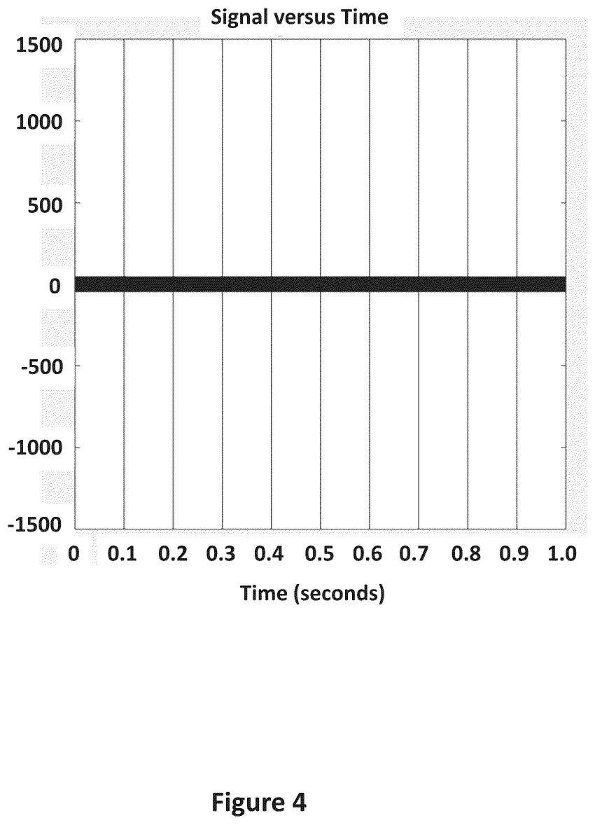

In some embodiments, ablation treatments comprise a combination of sequences with interleaved pulses, as shown for example in FIG. 4 and as following:

Sequence

| Parameter | A | C |
|---|---|---|
| Voltage [V] | 50 | 1500 |
| Frequency [kHz] | 35 | 35 |
| Positive Pulse width [μsec] | 1 | 2 |
| Negative Pulse width | 1 | 2 |
| Positive Pulse Amplitude | 50 | 1500 |
| Negative Pulse Amplitude | 50 | 1500 |
| Delay between pulses [μsec] | 0 | 0.5 |
| Number of Pulses in a burst | 1*10\hat{ }6 | 10*10\hat{ }6 |
| Number of bursts | 500000 | 10 |
| Delay between bursts [ms] | 0 | 100 |

### Example 4

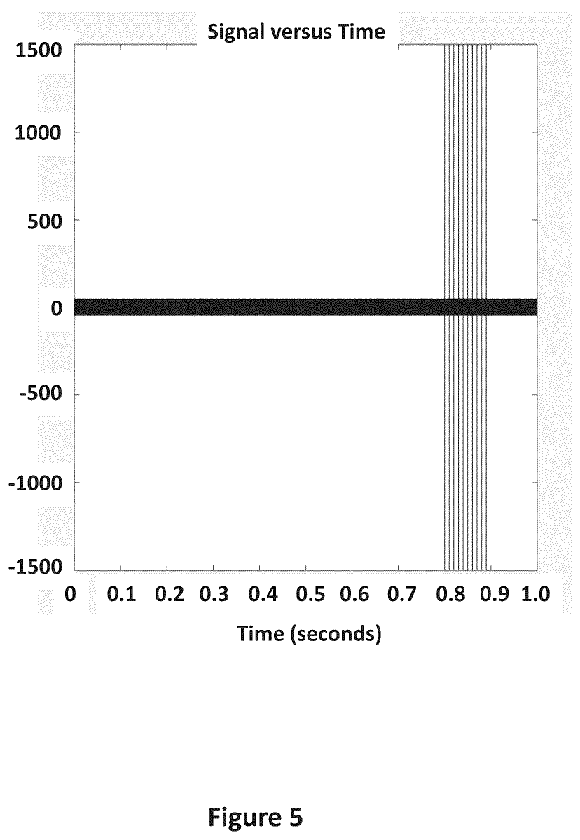

In some embodiments, ablation treatments comprise a combination of sequences with interleaved pulses, as shown example in FIG. 5 and as following:

Sequence

| Parameter | A | C |
|---|---|---|
| Voltage [V] | 50 | 1500 |
| Frequency [kHz] | 35 | 35 |
| Positive Pulse width [μsec] | 1 | 2 |
| Negative Pulse width | 1 | 2 |
| Positive Pulse Amplitude | 50 | 1500 |
| Negative Pulse Amplitude | 50 | 1500 |
| Delay between pulses [μsec] | 0 | 0.5 |
| Number of Pulses in a burst | 1*10\hat{ }6 | 20*10\hat{ }6 |
| Number of bursts | 500000 | 10 |
| Delay between bursts [ms] | 0 | 10 |

### Exemplary Treatment Tools (IRE Devices)

In some embodiments, electroporation treatments are performed using one or more of the following tools (IRE devices): cups, forceps, needles, probes and external electrodes.

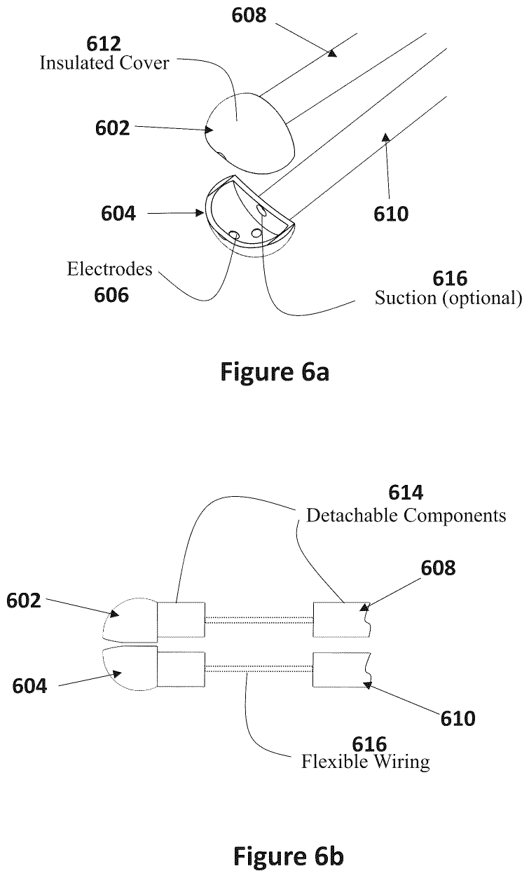

Referring now to FIGS. 6a and 6b, showing a schematic representation of an exemplary IRE device comprising electroporation cups, according to some embodiments of the invention. In some embodiments, the electroporation tools (IRE device) comprise two cups 602/604 comprising electrodes 606 in the internal surface of the cups, handles 608/610 to actuate the cups 602/604, and optionally an insulation cover 612 covering the external surface of the cups 602/604.

In some embodiments, the cups optionally comprise one or more detachable components 614, as shown for example in FIG. 6b. In some embodiments, the cups optionally comprise flexible wiring 616, as shown for example in FIG. 6b, which can potentially protect the patient from involuntary movements of the cups during the treatment. In some embodiments, one or more of the cups 602/604 optionally comprise an opening for suction 616, as shown for example in FIG. 6a.

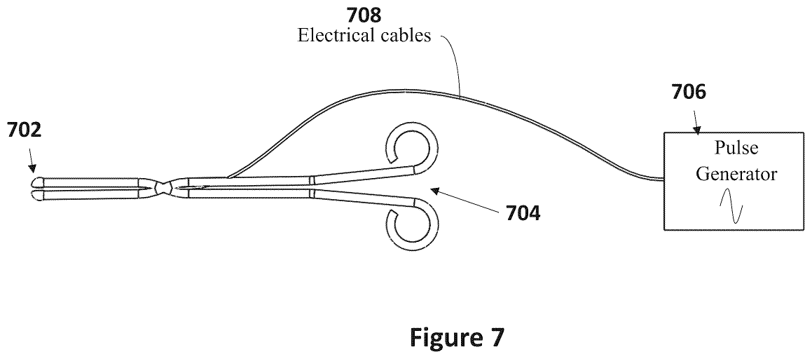

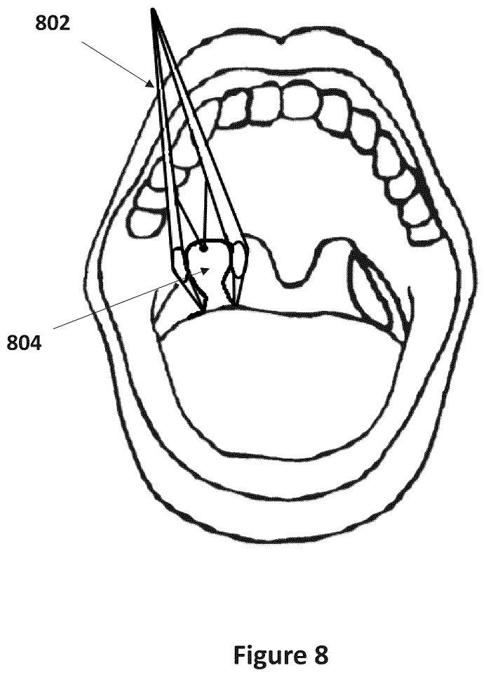

Referring now to FIG. 7, showing a schematic representation of electrode forceps, according to some embodiments of the invention. In some embodiments, the forceps comprise a distal end 702 for contacting the tissue. In some embodiments, the distal end 702 comprise cups 602/604, as shown for example in FIG. 6a and FIG. 6b. In some embodiments, the distal end are straight electrodes, which do not comprise cups. In some embodiments, the forceps comprise a handle 704 at the proximal end to manipulate the electrode, and schematically shown, the connection of the forceps to a pulse generator 706 by means of electrical cables 708.

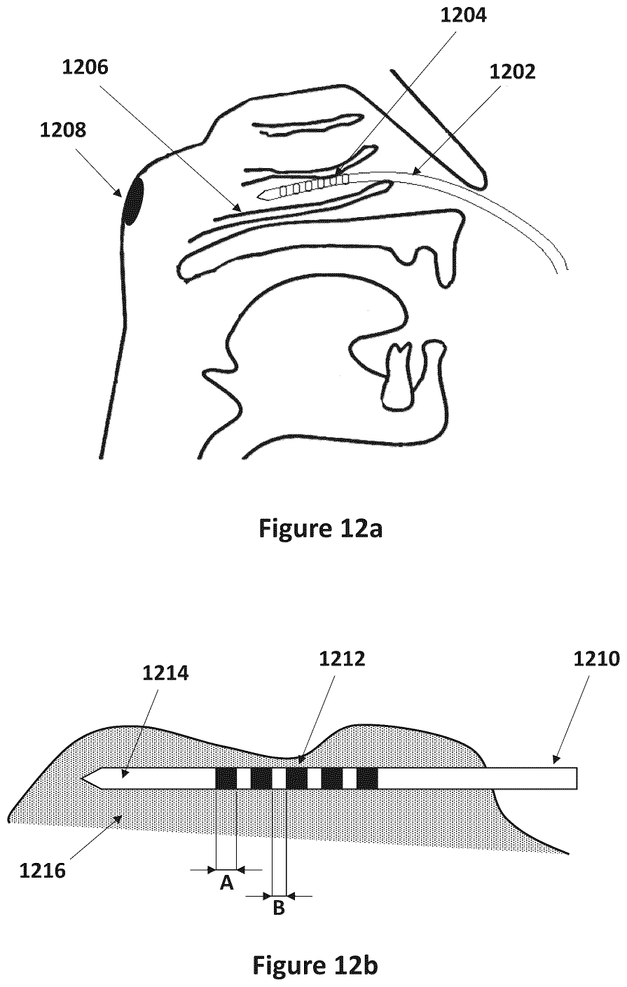

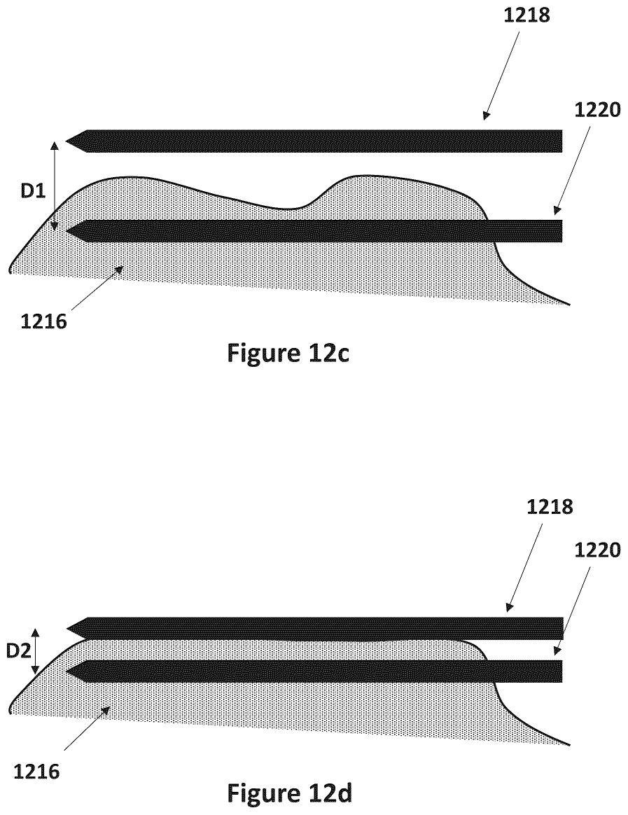

In some embodiments, electroporation treatments are performed using electrode needles (see for example text related to FIGS. 12a-d below). In some embodiments, the electrode needle optionally comprises both poles on the tip. In some embodiments, the electrode needle comprises one pole on the needle, and the other pole is located on an external (on the body, but in a remote location) electrode (see examples below).

In some embodiments, electroporation treatments are performed using electrode probes. In some embodiments, the electrode probe optionally comprises both/all poles on the tip. In some embodiments, the electrode probe comprises one pole on the probe and the other pole is located on an external (on the body, but in a remote location) electrode (see examples below). In some embodiments, the needle/probe comprises several electrodes.

In some embodiments, any of the abovementioned tools comprise one or more of the following safety features:

1. Indication of good contact between the electrode and the tissue before beginning the treatment;

2. The shape of the electrodes are as atraumatic as possible (incase a patient moves/jumps). In some embodiments, the device does not comprise sharp edges. In some embodiments, the device is flexible so as to allow movement of the device along with the patient's movements while holding well the area to be treated.

3. In some embodiments, the device comprises a flexible transmission arm that will absorb the patient's movement.

4. In some embodiments, in order to reduce the environmental damage to the surrounding tissue, during the procedure two cups will be used, which will pull the treated tissue. In some embodiments, the two cups will be insulated on the outside to reduce current leakage.

5. In some embodiments, short pulses will be provided to prevent the side effects of spasm.

6. In some embodiments, before performing the treatment, the level of contraction will be monitored—for example: first operation at 10-15 volts/3 Hz—for a few seconds to assess the reaction of the nerves in the area. In some embodiments, if the reaction is too strong, the position of the electrodes should be changed. In some embodiments, further examination is performed at 25% treatment voltage—400 volts—in some embodiments, the patient is anesthetized at this stage, no damage is shown but it does show the response of the nerves. In some embodiments, this is used to test if the treatment location is too close to the vagus or other sensitive tissues such as cardio arteries and/or veins.

7. In some embodiments, the system comprises hardware for monitoring the heart during the treatment. In some embodiments, the system uses a close loop heart-pounding feedback. In some embodiments, the system is configured to be sensitive to changes in the heart and detects for example if there is a change/extension of the heart activity (the distance between the intervals of the RR in the ECG) or in the heart rate—both in the experimental actions and in the treatment itself. In some embodiments, if a change that may impose a risk to the patient is detected the system will automatically and immediately stop the treatment. In some embodiments, for example at a detected change of above 50% in the ECG interval.

### Exemplary Treatment Locations

In some embodiments, electroporation treatments are performed for pathologies in one or more of the following locations: otorhinolaryngology related areas and the prostate. In some embodiments, any of the IRE devices disclosed herein can be used in either of those locations. In some embodiments, the operator chooses the type of IRE device to use according to the limitations and/or requirements of the tissue needed to be treated.

### Exemplary Treatments in Otorhinolaryngology Related Areas

In some embodiments, electroporation treatments are performed in otorhinolaryngology related areas to treat pathologies in one or more of the following zones: a) the tonsils such as pharyngeal tonsil, palatine tonsil; b) the adenoids; c) the base of the tongue; and d) the concha/turbine/inferior turbinate.

### Exemplary Treatments of the Tonsils

In some embodiments, treatment of the tonsils comprises reduction of tonsils mass or volume by utilizing electroporation without the need to extract them. In some embodiments, potential advantages of utilizing electroporation techniques, as disclosed herein, are significant reduction of treatment time—which can be as low as less than a minute (and can be even less than 30 seconds per tonsil) or a few minutes long; no open wound of the mucosa. There is actually no or minimal mucosal injury, so the recovery time is expected to be shorter; significant decrease in the risk of bleeding; and prevention or reducing of edema, swelling and inflammation, thus reducing pain and potentially preventing or reducing scarring. In some embodiments, the treatment on the tonsil will be intermittent, for example pulse-rest-pulse-rest, in a range of from about 1 second to about 30 seconds for each tonsil. In some embodiments, the preferred time is 10 seconds per tonsil.

Referring now to FIG. 8, showing a schematic representation of an electroporation treatment for the tonsils utilizing forceps, according to some embodiments of the invention. In some embodiments, an operator will utilize the forceps 802, comprising the electrodes, to provide electroporation treatment to the tonsils 804. In some embodiments, optionally, the tonsils will be held by another set of forceps or other shaped device with or without vacuum in order to isolate or keep away the tonsil from the surrounding tissue (not shown).

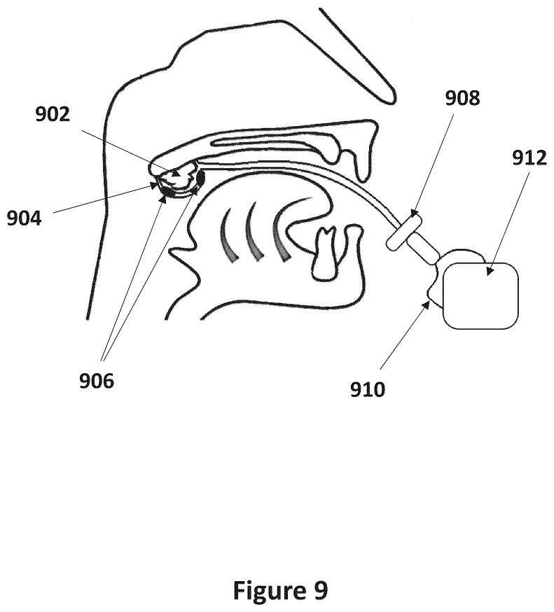

Referring now to FIG. 9, showing a schematic representation of an electroporation treatment for the tonsils utilizing a cup electrode, according to some embodiments of the invention.

In some embodiments, an operator will utilize the cup electrode 904, comprising the electrodes 906, to provide electroporation treatment to the tonsils 902. In some embodiments, the electrode cup comprises a handle 908 to be used to hold the cup electrode, which further comprises the cables 910 that communicate the cup electrode with the IRE generator 912. In some embodiments, as mentioned above, the cup electrode comprises a suction tube having an opening on the inside surface of the cup electrode (not shown). In some embodiments, such suction allows further separation from surrounding tissue reducing the chance of surrounding tissue involvement during electroporation treatment.

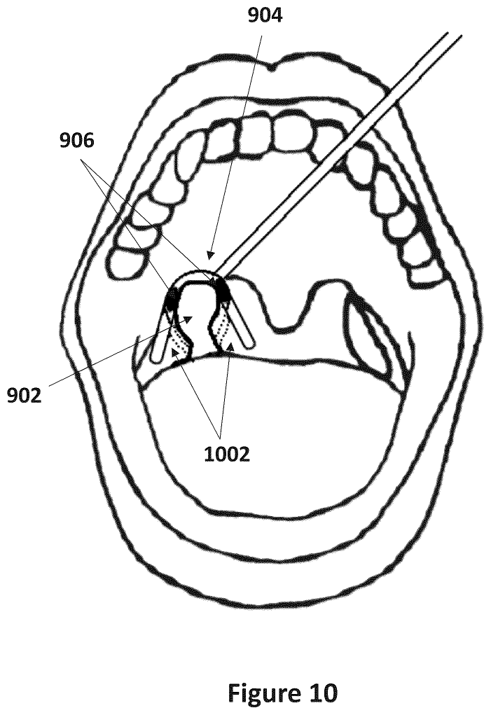

In some embodiments, the cup electrode comprises a closing mechanism 1002 that allows the user to further isolate the tonsil for the treatment, as schematically shown for example in FIG. 10 (relevant part numbers from FIG. 9 were kept for consistency). In some embodiments, a potential advantage of using a closing mechanism is that it allows to isolate the tonsil and potentially isolate the treated tissue to the tonsil tissue alone.

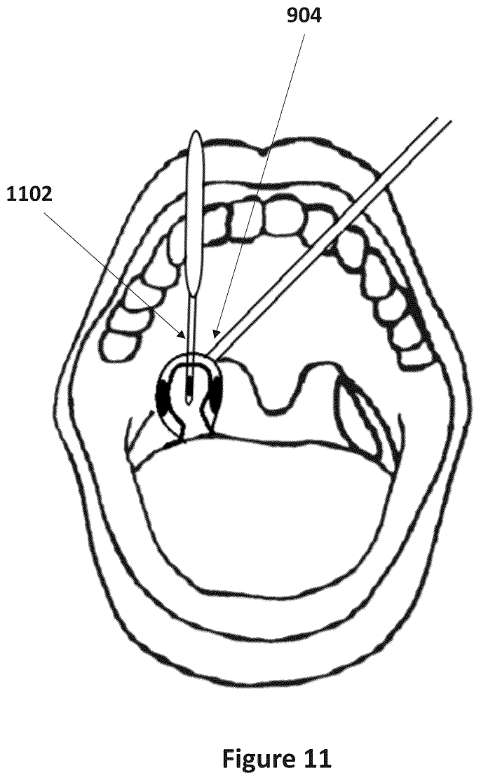

In some embodiments, a cup electrode is used in concomitance with one or more electroporation needles, as schematically shown in FIG. 11.

### Exemplary Treatments of the Base of the Tongue

In some embodiments, similarly to treatments for the tonsils, treatments for the base of the tongue are performed using any of the abovementioned devices and utilizing any of the abovementioned treatment protocols. In some embodiments, when treating location at the base of the tongue, two needles of about 2 to 3 mm thickness at a distance of from about 10 mm to about 20 mm from each other (optionally from about 7 mm to about 30 mm; Optionally from about 5 mm to about 50 mm), with either shallow or deep insertion of the needles into the tissue. In some embodiments, optionally, instead of two needles, it is used one needle inside the tissue and another on the surface of the tissue. In some embodiments, a plurality of needles are used, for example 5 or 6 needles that will conduct current between themselves at a predetermined sequence.

### Exemplary Treatments of the Inferior Turbinate/Adenoids

Referring now to FIG. 12a, showing a schematic representation of an electroporation treatment for the inferior turbinate/adenoids utilizing a probe or a needle, according to some embodiments of the invention. In some embodiments, an operator will utilize the probe/needle 1202, comprising two or more electrodes 1204, which will be operated between themselves in different sequences and combinations depending on the treated tissue, to provide electroporation treatment to the inferior turbinate 1206/adenoids 1208. In some embodiments, the probe/needle 1202 will be used to treat the inferior turbinate 1206/adenoids 1208. In some embodiments, a two-cup electrode is used to treat the excess tissue that is needed to be reduced.

Referring now to FIG. 12b, showing a schematic representation of an electroporation treatment for the inferior turbinate/adenoids utilizing one probe or one needle, according to some embodiments of the invention. In the following explanations, the word “probe” will be used to simplify the explanations, in each case it should be understood that it can be either a probe or a needle. In some embodiments, the IRE device comprises one probe 1210 with a plurality of electrodes 1212. In some embodiments, the probe 1202 comprises a single insulating element 1214 along with the plurality of electrodes 1212. In some embodiments, the electric field is created between adjacent electrodes, each electrode having a size A and a distance between electrodes is defined by a distance B. In some embodiments, the diameter of the electrode is from about 1 mm to about 4 mm. In some embodiments, the length of the electrode that is inserted within the tissue is from about 1 cm to about 6 cm. In some embodiments, the distance between the electrodes is from about 3 mm to about 150 mm. FIG. 12b shows the probe 1210 inside the tissue 1216.

Referring now to FIGS. 12c and 12d, showing a schematic representation of an electroporation treatment for the inferior turbinate/adenoids utilizing two probes or two needles, according to some embodiments of the invention. In some embodiments, the IRE device comprises two probes (or needles) 1218/1220. In the following explanations the word “probe” will be used to simplify the explanations, in each case it should be understood that it can be either two probes or two needles. In some embodiments, the probes are brought to the vicinity of the tissue (for example conchae). In some embodiments, a first probe penetrates the tissue while a second probe kept close to the first probe outside the tissue (meaning without penetrating the tissue). In some embodiments, both probes are inserted within the tissue. In some embodiments, the probes 1218/1220 are positioned in a longitudinal manner in relation to the tissue 1216, as shown for example in FIGS. 12c and 12d. In FIG. 12c probe 1218 is positioned outside the tissue 1216, while probe 1220 is located within the tissue 1216, and there is a distance D1 between them. In some embodiments, a hand-piece of the IRE device allows adjustment of the distance between the probes, as shown for example in FIG. 12d where the distance between a first probe 1218 and a second probe 1220 has changed from a distance D1 as shown in FIG. 12c to a distance D2 as shown in FIG. 12d. In some embodiments, adjusting the distance between probes allows full (or almost full) contact along the probes outside the tissue 1216 to generate a continuous electric field between the two probes. It should be understood that while the above explanations were provided with specific anatomical locations, the treatment can be used for other locations and/or pathologies, for example the base of the tongue for treating sleep apnea. Additionally, the treatment may comprise either the insertion of the electrodes into the tissue or the positioning of the electrodes outside the tissue without actually penetrating the tissue.

### Exemplary Prostate Treatments

In some embodiments, prostate electroporation treatments are used for the treatment of benign prostate enlargement (BPH). In some embodiments, prostate electroporation treatments comprise the use of an electrode inside the body of the patient and another electrode outside the patient. In some embodiments, insertion of an electrode inside the body of a patient is performed via to one or more of the following locations: via the urethra, via the perineum and via the rectum. In some embodiments, prostate electroporation treatments comprise the use of a voltage slightly higher than RF, but still slightly below the voltage used for IRE.

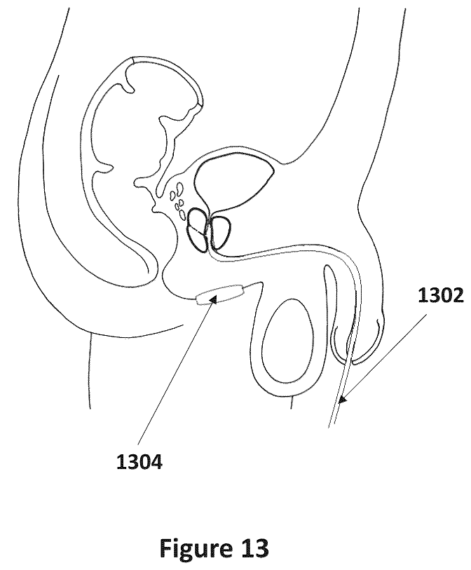

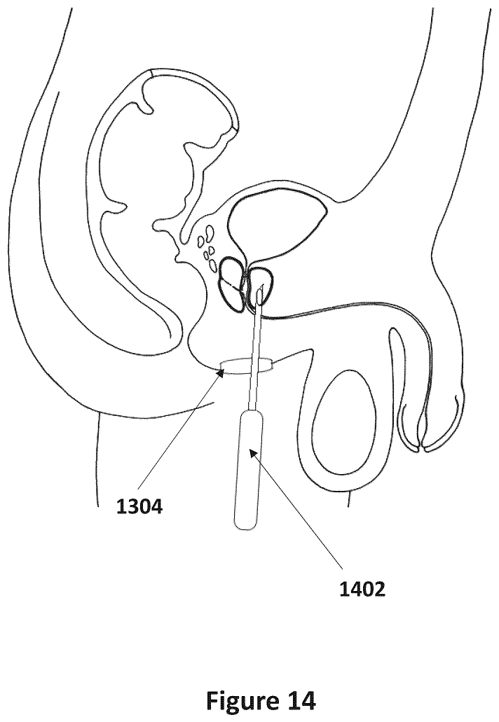

Referring now to FIG. 13, showing a schematic representation of a prostate treatment, according to some embodiments of the invention. In some embodiments, a first flexible electrode 1302 is inserted via the urethra while a second electrode 1304 is positioned outside, on the surface of the body of the patient.

Referring now to FIG. 14, showing a schematic representation of a prostate treatment, according to some embodiments of the invention. In some embodiments, a first needle electrode 1402 is inserted, for example, via the perineum while a second electrode 1404 is positioned outside, on the surface of the body of the patient.

### Exemplary Additional Applications of the Technology

In some embodiments, the technology disclosed herein can also be used for one or more of the following scopes:

1. Muscular uterus—In some embodiments, a superficial electrode mesh is placed on the inner uterine wall or needles are inserted into the uterine wall and electrophoresis is performed by passing a current between the needles/electrodes.

2. Neutering—in some embodiments, neutering treatments comprise the use of frequencies higher than RF to induce heating of the sperm duct while performing tissue sparing.

3. Hair loss—in some embodiments, treatments are performed to the follicle and/or the follicle area.

4. Fat—in some embodiments needles/electrodes are inserted in the tissue and, for example, ultra-high frequencies of tens of MHz are used. In some embodiments, optionally, additional electrolyte material is used during the treatment in order to increase the conduction in the tissue.

### General Technological Additions

In some embodiments, the device comprises visual means, for example a camera, to visualize the area of treatment before performing the treatment.

In some embodiments, the device comprises lighting means, for example a light, to allow better visualization of the area of treatment.

In some embodiments, in any of the abovementioned treatments, optionally, a gel and/or an electrolyte material is used to increase/improve the conduction, for example, a conductive liquid/gel or a non-conductive liquid/gel.

### Exemplary Treatment Procedures

Reference is now made to the following exemplary treatment procedures, which together with the above descriptions illustrate some embodiments of the invention in a non-limiting fashion.

In some embodiments, the procedure treatment involves delivering of series of intense but short pulses of energy through the probe electrodes, placed near the target tissue. In some embodiments, the short pulses are configured to generate an electrical field between the electrodes. In some embodiments, the generated field level is optimized to charge the cell membrane such that the transmembrane potential reaches a critical voltage level. In some embodiments, it is thought that the cell, trying to limit further temperature rise and prevent permanent damage, forms electrically conductive pores in the membrane. In some embodiments, if the pulses amplitude and duration are such that the cell cannot recover it results in tissue lesion without dependence on thermal processes to kill the cells. In some embodiments, the pulses amplitude and duration are evaluated and/or calculated so as to potentially avoid damage to the epithelium and/or the squamous epithelium and/or the respiratory epithelium. In some embodiments, the pulses amplitude and duration are evaluated and/or calculated so as to reduce tissue volume without damaging the epithelium. In some embodiments, the pulses amplitude and duration are evaluated and/or calculated so as to reduce tissue volume with a reduction of function of the epithelial tissue below five percent (reduction of function=<5%). In some embodiments, despite the use of very high electrical fields in order of 1000V/cm and higher, the thermal damage during the treatment is negligible due to the short duration (order of 100 microseconds) of the applied energy and the low repetition sequenced applied. In some embodiments, the total Joule heating is mitigated by the pulse duration, the number of pulses, specific sequence, and repetition frequency. In some embodiments, the ablation killing zone mainly depends on the induced electric field and tissue specific characteristics but not affected from tissue heat-sinks such as big blood vessels. In some embodiments, different cells such as nerve and blood vessels will have higher field thresholds for permanent damage. In some embodiments, due to the unique non-thermal effect of this ablation modality, the extracellular matrix is not affected by the treatment. In some embodiments, this enables tuning the ablation treatment to specific tissue cells and sparing of blood vessels and nerves.

In some embodiments, by applying bi-phasic and short duration pulses cells membrane charge is limited. In some embodiments, limiting the charging of the membrane prevents the cells action potential to reach its activation threshold. In some embodiments, this potentially significantly reduces nerve stimulation and muscles contraction.

In some embodiments, the treatment consists in using a probe having one or more electrodes with option to output an electrical signal from a first subset of electrodes to a second subset of electrodes and/or to another electrode located near the probe and/or outside of the body.

In some embodiments, high frequency IRE pulses potentially enable to generate energy with less potential for muscles contraction, less nerve stimulation effect, and more homogenous electrical field. In some embodiments, the treatment requires higher voltage for similar IRE effect like with low frequency IRE.

In some embodiments, an exemplary single sequence was optimized to minimize muscle contraction, nerve stimulation and with minimal or no thermal effect.

| | Sequence |
|---|---|
| Parameter | C |
| Voltage [V] | 2500 |
| Frequency [kHz] | 250 |
| Positive Pulse width [μsec] | 1.5 |
| Negative Pulse width | 1.5 |
| Positive Pulse Amplitude | 2500 |
| Negative Pulse Amplitude | 2500 |
| Delay between pulses [μsec] | 0.5 |
| Number of Pulses in a burst | 20*10\hat{ }6 |
| Number of bursts | 100 |
| Delay between bursts [ms] | 2 |

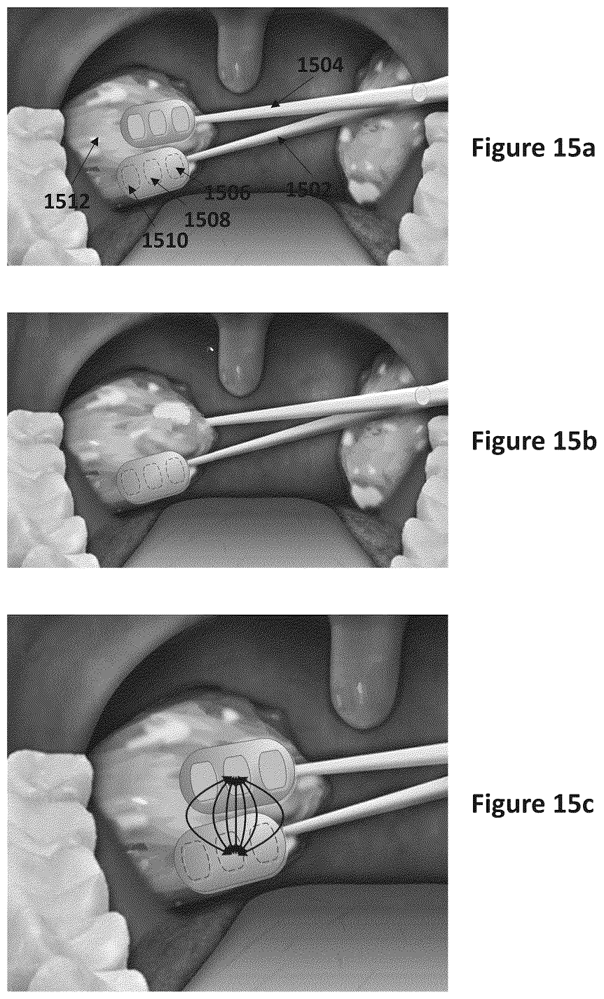

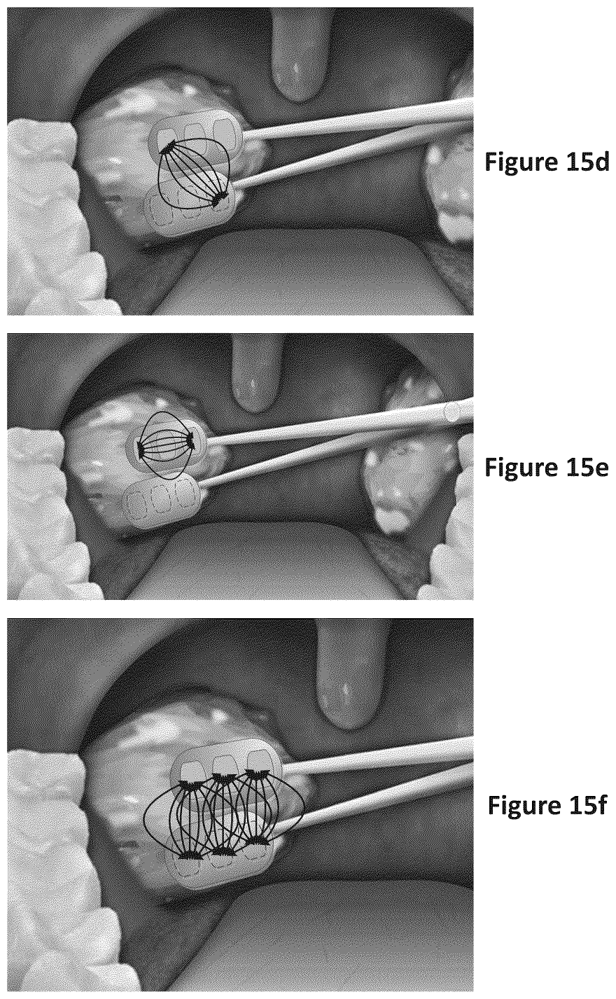

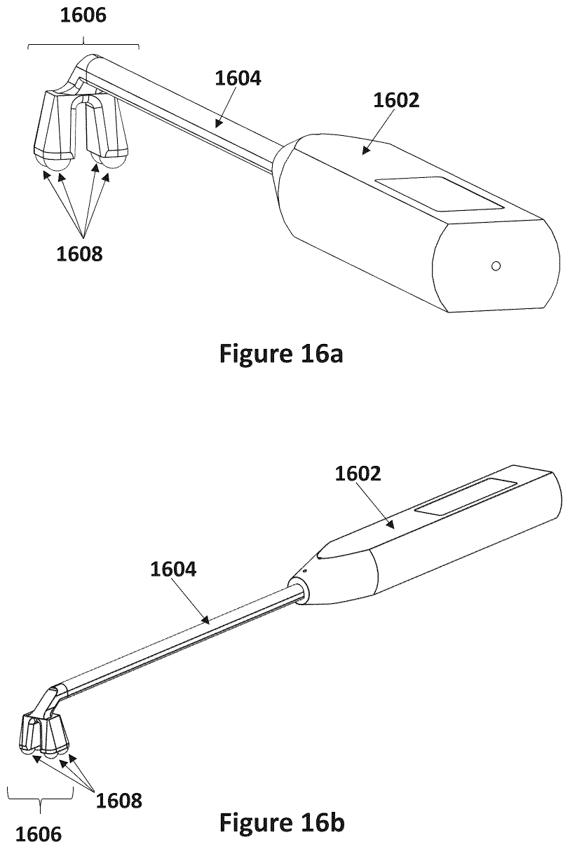

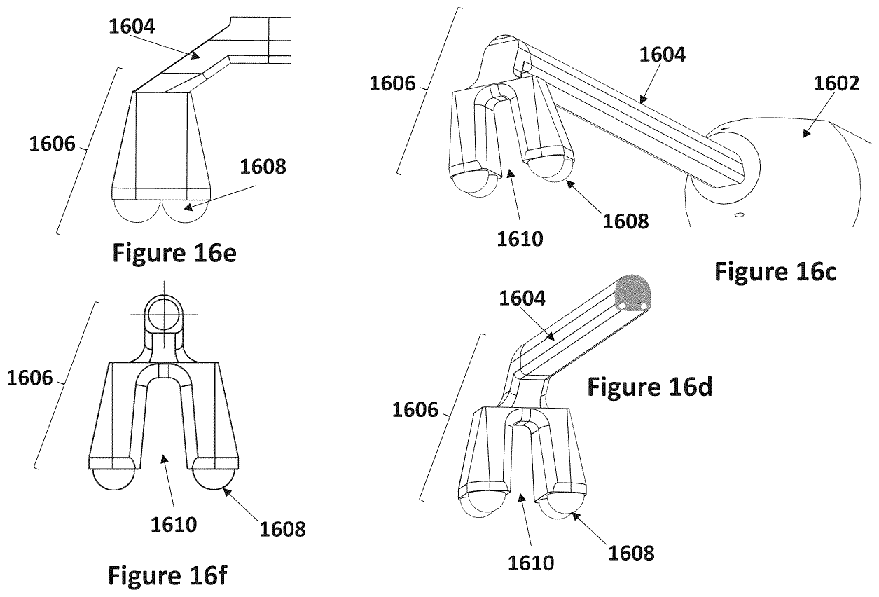

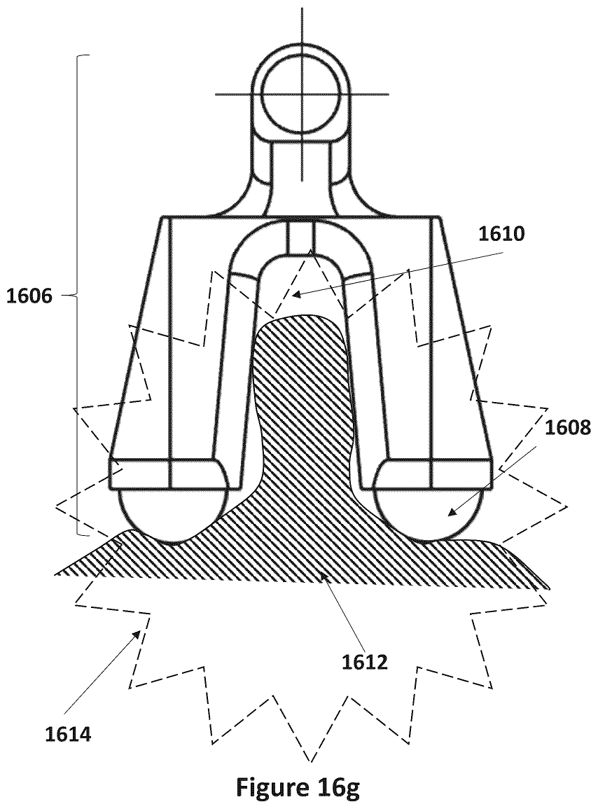

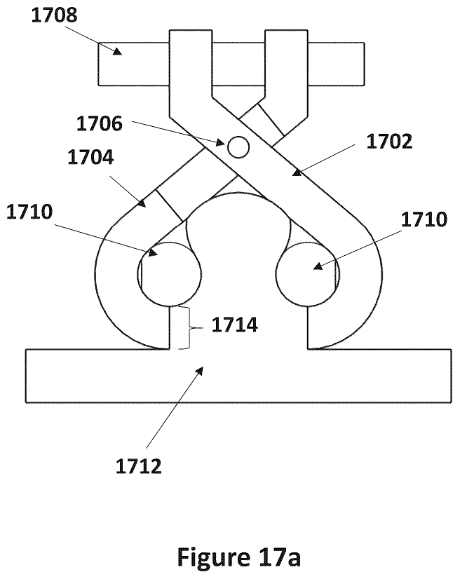

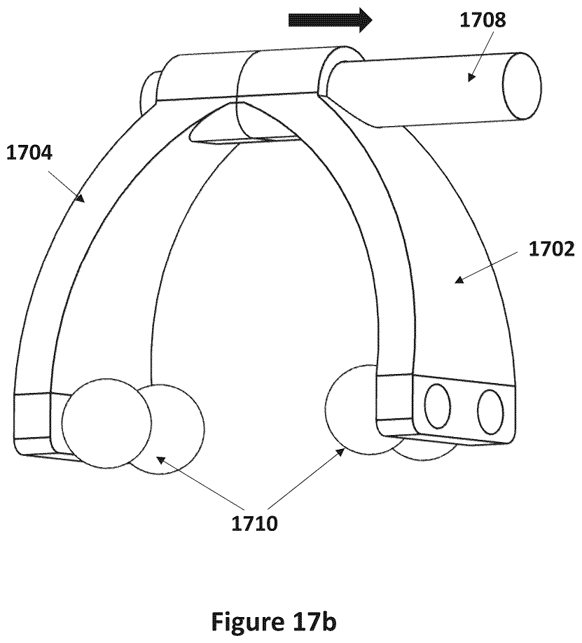

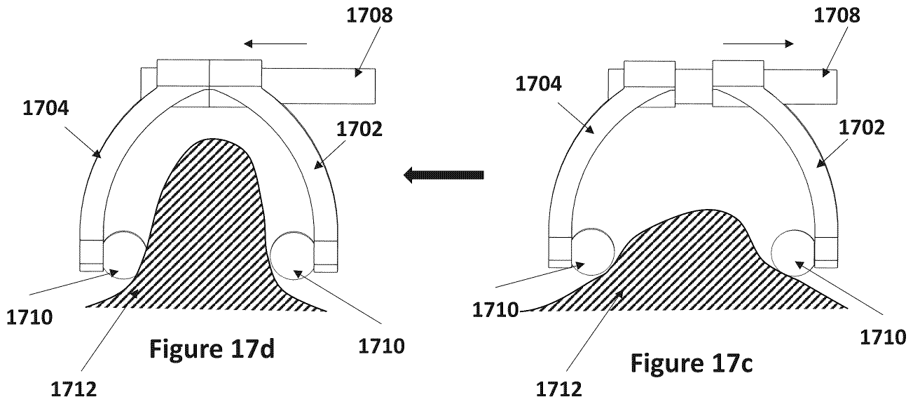

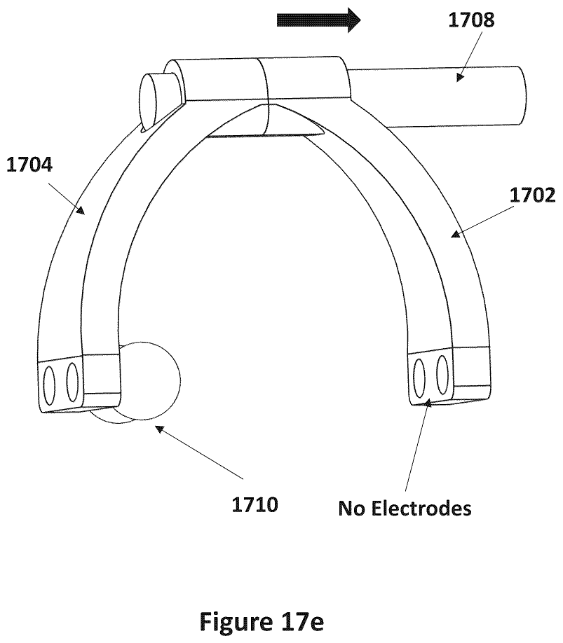

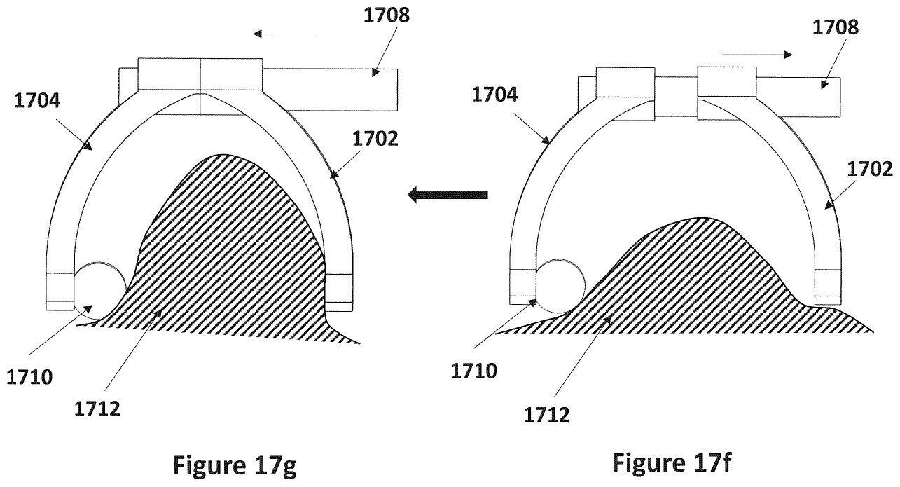

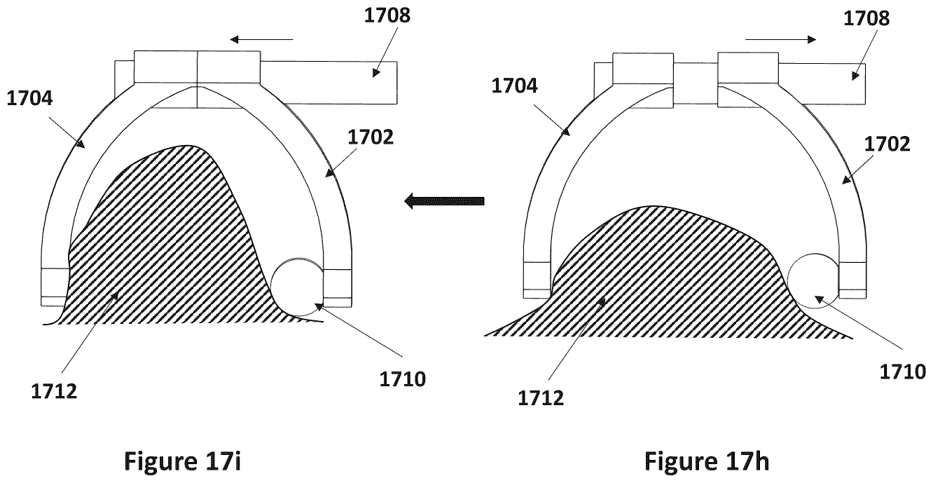

Referring now to FIGS. 15a-f, showing schematic representations of exemplary treatment of the tonsils, according to some embodiments of the invention. While the following exemplary treatment is explained using probes 1502/1504 it should be understood that other IRE devices can be used for the procedure, for example, the IRE devices as disclosed in FIGS. 16a-g and FIG. 17. In some embodiments, two probes 1502/1504 comprising three electrodes 1506/1508/1510 on each probe, are brought to the tonsil 1512 that is needed to be treated, as shown for example in FIG. 15a. In some embodiments, one probe is positioned on one side of the tonsil and the other on the other side of the tonsil, as shown for example in FIG. 15b. In some embodiments, current is passed between one electrode of one probe to another electrode of the other electrode, as shown for example in FIG. 15c. In some embodiments, current is passed between different electrodes than before, as shown for example in FIG. 15d. In some embodiments, current is passed between electrodes of the same probe, as shown for example in FIG. 15e. In some embodiments, current is passed between all the electrodes of one probe and all the electrodes of the other probe, as shown for example in FIG. 15f.

### Exemplary Structure of Handheld IRE Devices

Referring now to FIGS. 16a-g, showing schematic representations of an exemplary structure of exemplary handheld IRE devices, according to some embodiments of the invention. FIGS. 16a and 16b, show perspective views of exemplary handheld IRE devices according to some embodiments of the invention. In some embodiments, the exemplary handheld IRE device comprises a handle 1602 configured to be held by a user. In some embodiments, the handle 1602 comprises all the required electronics to provide IRE treatments. In some embodiments, the handle 1602 is connected, either wirelessly and/or by wire to a dedicated computing device configured to provide the necessary instructions for the IRE treatment. In some embodiments, the handle 1602 comprises a power source. In some embodiments, the handle 1602 receives the power from an external source. In some embodiments, the exemplary handheld IRE device further comprises an elongated body 1604, comprising a proximal end and a distal end, the proximal end is in mechanical and continuous communication with the handle 1602. In some embodiments, the elongated body 1604 is configured to house electrical wires (not shown) used to activate the electrodes located at a distal end of the handheld IRE device. In some embodiments, the wires run from the handle 1602 at the proximal end of the handheld IRE device, internally through the elongated body 1604 until the electrodes located at the distal end of the handheld IRE device. In some embodiments, the elongated body 1604 is configured to reversibly bend to allow better access to the tissue needed to be treated. In some embodiments, the exemplary handheld IRE device further comprises an operational distal end 1606 comprising one or more electrodes 1608. In the examples shown in FIGS. 16a and 16b, there are four electrodes. It should be obvious for a person having skills in the art that a different number of electrodes can be used, for example: 2, 6, 8, 10, 12, etc. A different number of electrodes are also part of embodiments of the invention.

Referring now to FIGS. 16c, 16d and, 16e 16f, showing schematic representations of different views of an exemplary operational distal end 1606 of an exemplary handheld IRE device, according to some embodiments of the invention. In some embodiments, the operational distal end 1606 comprises one or more grooves 1610 defined by a space between electrodes 1608. In the FIGS. 16c-f only one groove 1610 is shown. In some embodiments, the groove 1610 allows tissue 1612 to enter the space within the groove, as schematically shown, for example, in FIG. 16g. In some embodiments, the configuration of the operational distal end 1606 with the groove 1610 allows the tissue to “flow” within the space while maintaining electrical and mechanical contact between the electrodes 1618 and the treated tissue 1612. In some embodiments, the spatial electric field 1614 (dashed starred figure) creates the anticipated E-filed required to create the IRE effect in the volume including the space under the electrodes 1608 and the volume trapped between the electrodes 1618 inside the groove 1610. In some embodiments, the groove 1610 comprises a width that varies between 1 mm and 15 mm, optionally between 0.5 mm and 20 mm, optionally between 0.1 mm and 30 mm; and comprises a height that varies between 1 mm and 15 mm, optionally between 0.5 mm and 20 mm, optionally between 0.1 mm and 30 mm. In some embodiments, different sizes of operational distal ends (electrodes and/or grooves) can be used to treat different sizes of tissues in different locations, optionally locations with limited access (for example within the nose). In some embodiments, the dimension of the groove varies and/or can be varies (extended or retracted) in accordance with the volume of the tissue that is needed to be treated (see below). In some embodiments, a potential advantage of providing the possibility to change the distance between electrodes and/or the possibility to change the size of the groove is that it potentially allows to use the best configuration of IRE device and IRE protocols for the specific tissue needed to be treated.

In some embodiments, the volume of the tissue is determined using one or more of the following techniques:
Mechanically measuring the width and height of the tissue and calculating the volume of a ball assuming either the average radium obtained from the width and height or taking either width or height as ball radius;
Using a single-camera system which captures high-resolution 3D images in one shot. In some embodiments, the camera is inserted into the treatment area (for example the mouth) allowing volume measurement of the tissue (for example: tonsil, adenoid and/or base of tongue volume).

In some embodiments, the IRE devices are configured to be operated by a single user, without the need of assistance from a secondary operator and/or nurse and/or physician.

In some embodiments, the operational distal end can be replaced to match the requirements of the tissue needed to be treated, for example, a smaller operational distal end may be needed to treat the adenoids, when compared to the operational distal end required to treat the tonsils.

Referring now to FIG. 17a, showing a schematic representation of an additional exemplary handheld IRE device, according to some embodiments of the invention.

In some embodiments, the IRE device is configured to allow the user to grab the tissue that will be treated. In some embodiments, the IRE device is similar to that disclosed in FIGS. 16a-16g, but with a different configuration of the operational distal end. In some embodiments, the operational distal end of the IRE gripping device comprises two arms 1702/1704, attached to each about a center location by a pivot 1706. In some embodiments, the arms 1702/1704 are attached to a slider 1708 on the proximal end. In some embodiments, on each distal end of each arm there is at least one electrode 1710. In some embodiments, the electrode 1710 comprises a spherical form. In some embodiments, when the IRE device is positioned over the required tissue 1712, the operator actuates the sliders 1708, thereby spatially grabbing the tissue. In some embodiments, when the arms reach the required distance or a predetermined distance between the electrodes 1710 the user may activate the IRE protocol. In some embodiments, the IRE device further comprises means to notify the user of the movement of the arms, for example, by mechanical means such as a mechanical “click” sound. In some embodiments, at each certain distance between electrodes, the system is required to provide a specific IRE treatment protocol. In some embodiments, the system is configured to automatically assess the distance between electrodes (either mechanically or electrically) and automatically modify the IRE treatment protocol accordingly. For example, when the distance between the electrodes is changed the impedance between the electrodes is changed. In some embodiments, the device measures the impedance when electrodes contact the treated tissue and signals the user when operation conditions (for example, when the impedance is from about 80 ohm to about 120 ohm, which provides the appropriate operating conditions for the device) are achieved. In some embodiments, when impedance is out of the range the device indicates the user that the system cannot be operated under the current conditions. In some embodiments, the system comprises a dedicated algorithm configured to automatically amend the characteristic of the pulse (for example: voltage, frequency, number of pulses) as a function of the distance between the electrodes. In some embodiments, the distance between electrodes is measured using a linear hall sensor, or and encoder (optical, mechanical) that measures the actual distance between the electrodes and then the system calculates the optimal system operation parameters to obtain the anticipated (and required) electrical fields. In some embodiments, additionally or alternatively, electrical means such as continuous electrical impedance measurement between the electrodes provides a precise indication of the minimal and optimal gripping force required for optimal system operation. In some embodiments, the impedance may be indicated by sound, visual indication, or any combination of the two. In some embodiments, a potential advantage of the spherical electrodes is that it potentially increases the insulation effect of tissues that are not needed to be affected by the system electric fields generated during the treatment, and this is due the geometrical properties of the gripping elements. In some embodiments, the electrodes 1710 are positioned along a curved structure made of non-conductive material. In some embodiments, the insulating material comprises a thickness 1714 aimed to create a distance between the electric fields generated by the device and the tissues that are not aimed to be affected by the device. In some embodiments, the protection from the electric fields can be determined as a function of the calculated electric field generated by the electrodes (electric fields are correlated to the electrode's shape and the distance between the electrodes besides system electrical characteristics). In some embodiments, the insulation material thickness can vary between 0.1 mm to 15 mm.

### Potential Advantages of IRE Device

In some embodiments, the IRE device is configured to allow the user to treat a wide range of sizes and morphologies of tissues, for example, tonsils and adenoids. In some embodiments, additionally the IRE device comprises dedicated protective features configured to potentially avoid damaging tissues that are not intended to receive the IRE treatment, meaning protective features configured to protect tissues from IRE electric fields in the vicinity of the treatment area (for example: within the mouth), tissues that are not intended to be affected during the IRE procedure. In some embodiments, the IRE device is configured to allow optimally mechanical and electrical interface between the electrodes and the treated tissue by applying local compression force on the soft tissue. In some embodiments, by applying the gripping force on the soft tissue, the organ will better comply with the electrode's geometric properties. In some embodiments, the architecture of the IRE device potentially reduces the risk of human errors to create an electrical interface between the electrodes and the treated tissue.

In some embodiments, the mechanism that allows the user to set a distance between the electrodes is based on a rotation as shown for example in FIG. 17. In some embodiments, the mechanism that allows the user to set a distance between the electrodes is based on linear translation of the electrodes by using, for example, sliders or linear rails, as shown for example in FIG. 17b (same part have same numbers). FIGS. 17c and 17d show a schematic representation of the IRE device as shown in FIG. 17b, grabbing the tissue 1712. FIG. 17c shows the device in an open configuration, and FIG. 17d shows the device in a closed configuration grabbing the tissue. In some embodiments, the IRE device can have electrodes on one arm, as shown for example in FIG. 17e. In some embodiments, in these cases, one side is used to grab the tissue and push it towards the electrodes located on the other arm. While the device shown in FIG. 17e shows the electrodes on arm 1704, it should be understood that the electrodes can be on either arm. FIGS. 17f, 17g, 17h and 17i show schematic representations of the IRE device having electrodes on one arm, grabbing the tissue 1712. FIGS. 17f and 17h show the device in an open configuration, and FIGS. 17g and 17i show the device in a closed configuration grabbing the tissue.

In some embodiments, not shown in the figures, the movement mechanism of one or both arms is performed by using a hinge located in the connection between the arm and the elongated body. In some embodiments, the hinge can be in one of the arms or in both arms. In some embodiments, the hinge is conjured to allow movement of the arms towards each other to allow gripping of the tissue between the arms.

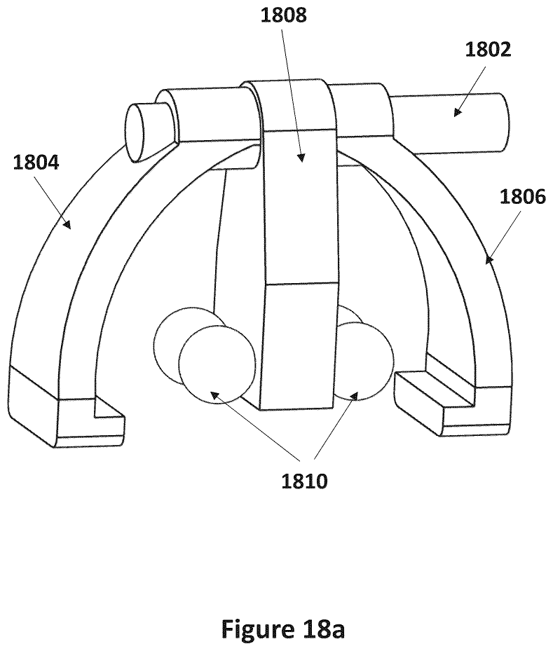

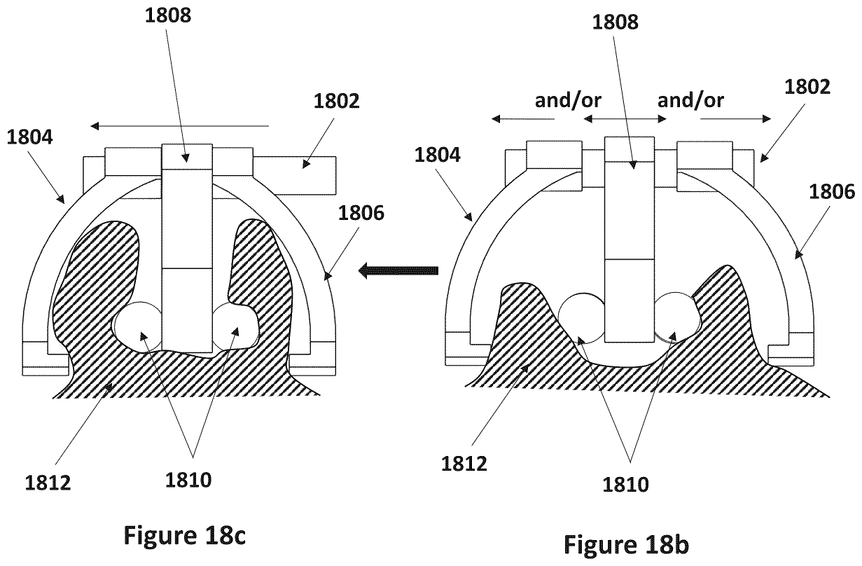

Referring now to FIG. 18a, showing a schematic representation of an additional exemplary handheld IRE device, according to some embodiments of the invention. In some embodiments, the IRE device comprises an elongated body 1802, similar to those disclosed above, a distal arm 1804, a proximal arm 1806 and a middle element 1808 comprising the electrodes 1810. In some embodiments, distal arm 1804 and proximal arm 1806 act as a gripping mechanism, for example by linearly moving the proximal arm 1806 either distally or proximally, in relation to the distal arm 1804. In some embodiments, the gripping mechanism is configured to compress the tissue over the electrodes 1810 located on the middle element 1808. In some embodiments, the electrodes are positioned at a predetermined distance from each other. In some embodiments, a potential advantage of this IRE device is that it potentially allows a more flexible operation of the system depending on the organ (tonsil, adenoid, base of tongue, concha) morphology and sizes. FIGS. 18b and 18c show a schematic representation of the IRE device as shown in FIG. 18a, grabbing the tissue 1812. FIG. 18b shows the device in an open configuration, and FIG. 18c shows the device in a closed configuration grabbing the tissue 1812. In some embodiments, either one arm moves and the other arm and the middle element remain static, or any of the arms and the middle element can move. In some embodiments, only the arms can move.

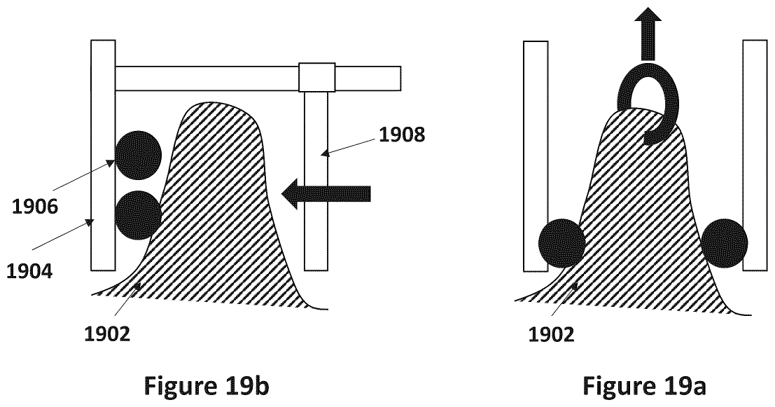

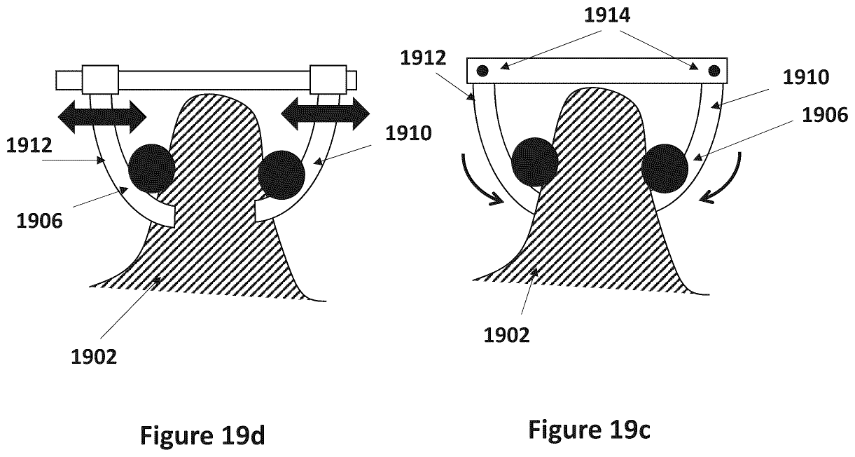

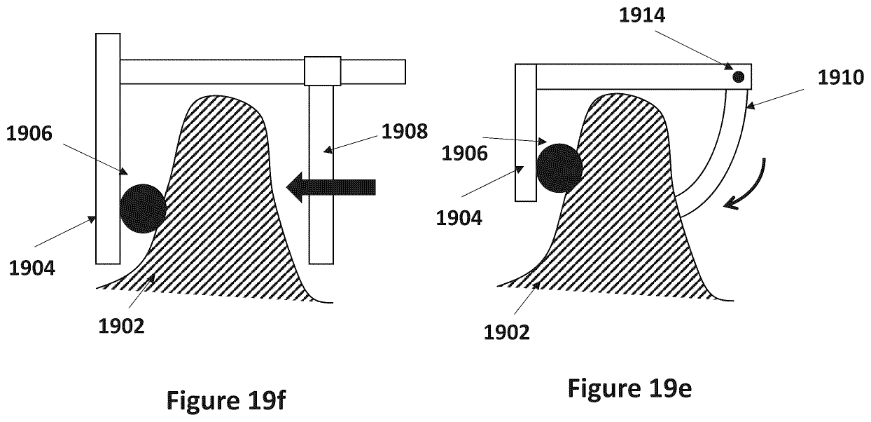

In general, any of the IRE devices disclosed therein are configured to grab the tissue 1902 and enable providing local IRE treatment to the chosen tissue. For example, in some embodiments, the grabbing can be done by holding the tissue 1902 and contacting the tissue 1902 from both sides, as shown for example in FIG. 19a. For example, in some embodiments, the grabbing can be done by having a static element 1904 comprising the electrodes 1906 and a linearly mobile element 1908 that brings the tissue towards the electrodes 1906, as shown for example in FIG. 19b. For example, in some embodiments, the grabbing is performed by two arms 1910/1912 comprising electrodes 1906 connected by hinges 1914, in which the arms are brought towards the tissue 1902 angularly, as shown for example in FIG. 19c. In some embodiments, the arms are brought towards the tissue using a linear movement, as shown for example in FIG. 19d. For example, in some embodiments, the grabbing can be done by having a static element 1904 comprising the electrodes 1906 and an arm 1910 without an electrode connected with a hinge 1914, as shown for example in FIG. 19c. In some embodiments, as previously disclosed, there can be any number of electrodes, FIG. 19f is similar to FIG. 19b, but with less electrodes.

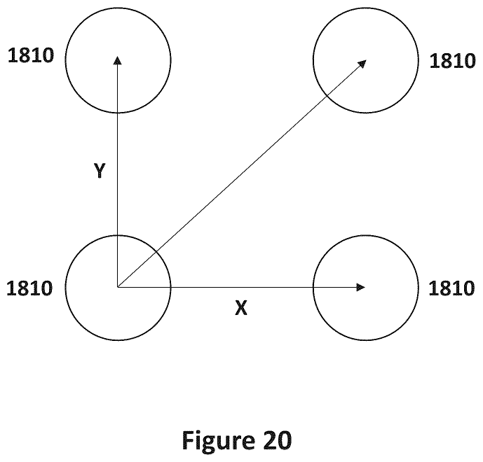

In some embodiments, for example, the electrode configuration of the IRE system is as shown, for example, in FIG. 20. In some embodiments, the distance between the electrodes is presented as vector norm D=√(x²+y²) which is constant. Therefore, the IRE system is configured to amend the parameters of activation so as to maintain a constant electric field in view of the specific, and possibly changing, positions of the electrodes. In some embodiments, the distance between the electrodes is set by the user before the procedure. In some embodiments, the system comprises a plurality of preassembled middle elements 1808 having electrodes arranged differently, which a user can choose according to the specific needs of the IRE procedure.

In some embodiments, the user sets the IRE system operation parameters based on a predetermined norm or the system can auto-calculate the pulse characteristics in accordance with the distances between electrodes as presented by a vector between each electrode in the array of electrodes. In some embodiments, the system may comprise any number of electrodes, for example, 2, 3, 4, 10, 20, etc., electrodes.

As used herein the term “about” refers to ±20%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of reducing volume of a tissue, the method comprising:
a. setting one or more IRE parameters;
b. inserting an IRE device within a zone of a mouth or a nose;
c. enclosing an area of interest of said tissue within a space between at least two electrodes;
d. applying irreversible electroporation treatment according to said set IRE parameters.

2. The method according to claim 1, wherein said irreversible electroporation treatment comprises at least one sequence comprising a frequency higher than 5 kHz.

3. The method according to claim 1, wherein said applying comprises non-thermally applying said irreversible electroporation treatment.

4-7. (canceled)

8. The method according to claim 1, wherein said treatment is a non-invasive treatment.

9. The method according to claim 1, further comprising applying two or more sequences in said treatment.

10. The method according to claim 2, wherein said at least one sequence comprises the following parameters:

| | Sequence |
|---|---|
| Parameter | A |
| Voltage [V] | 0-400 |
| Frequency [kHz] | 20-500 |
| Positive Pulse width [μsec] | 1-20 |
| Negative Pulse width [μsec] | 1-20 |
| Positive Pulse Amplitude [V] | 0-400 |
| Negative Pulse Amplitude [V] | 0-400 |
| Delay between pulses [μsec] | 0-8 |
| Number of Pulses in a burst | 0-60*10\hat{ }6 |
| Number of bursts | 0-100000 |
| Delay between bursts [ms] | 0-5000 |

11. The method according to claim 2, wherein said at least one sequence comprises the following parameters:

| | Sequence |
|---|---|
| Parameter | B |
| Voltage [V] | 100-2500 |
| Frequency [kHz] | 5-40 |
| Positive Pulse width [μsec] | 10-100 |
| Negative Pulse width [μsec] | 10-100 |
| Positive Pulse Amplitude [V] | 100-2400 |
| Negative Pulse Amplitude [V] | 100-2400 |
| Delay between pulses [μsec] | 0-150 |
| Number of Pulses in a burst | 0-20*10\hat{ }6 |
| Number of bursts | 0-100000 |
| Delay between bursts [ms] | 0-1000 |

12. The method according to claim 2, wherein said at least one sequence comprises the following parameters:

| | Sequence |
|---|---|
| Parameter | C |
| Voltage [V] | 400-3000 |
| Frequency [kHz] | 10-500 |
| Positive Pulse width [μsec] | 1-5 |
| Negative Pulse width [μsec] | 1-5 |
| Positive Pulse Amplitude [V] | 400-3000 |
| Negative Pulse Amplitude [V] | 400-3000 |
| Delay between pulses [μsec] | 0-50 |
| Number of Pulses in a burst | 0-40*10\hat{ }6 |
| Number of bursts | 0-100000 |
| Delay between bursts [ms] | 0-1000 |

13. A device for performing irreversible electroporation treatment to a subject, comprising:
a. a handle, comprising a first proximal end and a first distal end;
b. an elongated body comprising a second proximal end and a second distal end; said second proximal end being in mechanical communication with said first distal end of said handle;
c. an operational distal end comprising at least two electrodes distanced from each other, said at least two electrodes sized and shaped to receive therein at least one tissue in need to receive said irreversible electroporation treatment;
d. a computing device comprising instructions to provide said irreversible electroporation treatment at parameters characterized by causing a minimal damage to epithelial tissue.

14. The device according to claim 13, wherein each of said at least two electrodes comprise a concave shape or a shape adaptive to the treated organ surface located at said second distal end of said elongated body.

15-17. (canceled)

18. The device according to claim 13, wherein said at least two electrodes comprise an architecture that expands the electrical field to a wide volume.

19-20. (canceled)

21. The device according to claim 13, further comprising at least one isolation material covering at least part of said operational distal end.

22. The device according to claim 13, wherein said elongated body is configured to be bent.

23-55. (canceled)

56. The device according to claim 13, wherein said parameters are configured for allowing using said device without using any kind of intubation.

57. The device according to claim 13, further comprising a mask configured to provide one or more of an anaesthetic and oxygen.

58. The device according to claim 13, wherein said computing device comprises instructions for amending IRE parameters so as to maintain a constant electric field in view of the positions of the electrodes.

59. The device according to claim 13, wherein said device is further configured for measuring a gripping force between said at least two electrodes.

60. The method according to claim 1, wherein said method is configured to reduce volume of said tissue without damaging epithelial tissue.

61. The method according to claim 1, wherein said method is performed without intubation of a patient.

62. The method according to claim 1, further comprising inducing a limited heat at said zone.

63. The method according to claim 1, wherein said one or more IRE parameters are characterized by causing minimal damage to epithelial tissue.

64. The method according to claim 1, further comprising providing a mask; and providing one or more of an anaesthetic and oxygen via said mask.

65. The method according to claim 1, further comprising amending said one or more IRE parameters so as to maintain a constant electric field in view of the positions of the electrodes.

66. The method according to claim 1, further comprising measuring a gripping force between said at least two electrodes.

67. A method of reducing volume of a tissue, the method comprising:
a. inserting an IRE device within a zone of a mouth or a nose;
b. enclosing an area of interest of said tissue within a space between at least two electrodes;
c. applying irreversible electroporation treatment;
wherein the tissue is selected from the group consisting of:
i. tonsils;
ii. adenoids;
iii. a base of the tongue; and
iv. concha/turbine/inferior turbinate.

68. A method of reducing volume of a tissue in a throat, the method comprising:
a. providing two or more external electrodes configured for performing irreversible electroporation treatment;
b. selecting one or more IRE parameters;
c. enclosing an area of said throat within a space between said at least two external electrodes;
d. applying irreversible electroporation treatment according to said selected IRE parameters.

69. A device for performing irreversible electroporation treatment to a throat of a subject, comprising:
a. a handle, comprising a first proximal end and a first distal end;
b. an elongated body comprising a second proximal end and a second distal end; said second proximal end being in mechanical communication with said first distal end of said handle;
c. an operational distal end comprising at least two external electrodes separated from each other, said at least two external electrodes sized and shaped to receive therein an area of a throat in need to receive said irreversible electroporation treatment;
d. a computing device comprising instructions to provide said irreversible electroporation treatment at parameters characterized by causing a minimal damage to epithelial tissue.
